(19) United States
(12) Patent Application Publication
(10) Pub. No.: US 2011/0003879 A1
(43) Pub. Date: Jan. 6, 2011

(54) Antisense oligonucleotides targeted to the coding region of thymidylate synthase and uses thereof

(76) Inventors: Mark D. Vincent, Ontario (CA); D. James Koropatnick, Ontario (CA)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/908,389

(22) PCT Filed: Mar. 13, 2006
(86) PCT No.: PCT/CA06/00350

§ 371 Date: Sep. 17, 2010

Related U.S. Application Data
(60) Provisional application No. 60/661,993, filed on Mar. 11, 2005.

(51) Int. Cl. A61K 31/7088; C07H 21/04; A61P 35/00

(52) U.S. Cl. 514/44 A; 536/24.5

(57) ABSTRACT

Antisense oligonucleotides directed to the coding region of a mammalian thymidylate synthase mRNA that are capable of inhibiting the proliferation of cancer cells without decreasing the level of thymidylate synthase mRNA in the cells are provided. The antisense oligonucleotides are also capable of inducing apoptosis in the cancer cells. The antisense oligonucleotides can be used to inhibit the proliferation of cancer cells and to induce apoptosis in cancer cells. Methods of treating cancer, and in particular breast cancer, with the antisense oligonucleotides, alone or in combination with other therapeutics, are also provided.

### FIELD OF THE INVENTION

The present invention pertains to the field of cancer therapeutics and in particular to antisense oligonucleotides for the treatment of cancer.

### BACKGROUND

A number of proteins have been implicated in cancer and, as a result, are targeted by standard chemotherapeutics for cancer treatment. An example of such a protein is thymidylate synthase (TS), which is an essential enzyme in de novo production of thymidylate (Carreras and Santi, 1995, Annu. Rev. Biochem. 64:721-762). Due to the crucial role of TS in DNA synthesis and cell proliferation, it has been an important target for cancer chemotherapy for many years (Danenberg, 1977, Biochim. Biophys. Acta 473:73-92; Danenberg et al., 1999, Semin. Oncol. 26:621-631).

Many agents have been developed to target TS and its enzymatic activity, including 5-fluorodeoxyuridine (5-FUdR), raltitrexed, and pemetrexed (Danenberg P V, et al., Oncol 1999; 26:621-631; Chu E, et al., Cancer Chemother Pharmacol 2003; 52 Suppl 1:S80-89). Targeting TS has proven useful in the treatment of head and neck, breast and colorectal cancers and mesothelioma (Mirjolet J F, et al., Br J Cancer 1998; 78:62-68; Lehman N L. Expert Opin Investig Drugs 2002; 11:1775-1787; Rose M G, et al., Clin Colorectal Cancer 2002; 1:220-229).

Antisense oligonucleotides, in the form of antisense RNA, targeted at the translation site at the 5′ end of thymidylate synthase mRNA have been described (Ju, J., “Increased Drug Sensitivity of Tumor Cells by Manipulation of Cell Cycle Control Genes” 1996, Thesis University of Southern California, pp. 100-130). Ju attempted to use short antisense oligonucleotides to increase cytotoxicity of HT-29 cells to FdUrd, however, these attempts were unsuccessful until larger RNA antisense oligonucleotides were used. Antisense oligonucleotides that target and impact upon the expression of TS mRNA have also been described in U.S. Pat. No. 6,087,489; International Patent Applications WO 99/15648 and WO 98/49287.

A specific antisense oligonucleotide targeting the 3′-untranslated region of TS mRNA has been shown to downregulate TS mRNA and protein and sensitize human HeLa cervical carcinoma and HT-29 colon tumour cells to 5-FU, 5-FUdR and raltitrexed in tissue culture and in immunocompromised mice (Berg et al., 2001, J. Pharmacol. Exp. Ther. 298:477-484). More recently, the use of this antisense oligonucleotide to increase the sensitivity of cells that over-express TS to 5-FUdR has been demonstrated (Ferguson, et al., 2001, Br. J. Pharmacol. 134:1437-1446). We previously reported that treatment of human cervical carcinoma (HeLa) cells with an antisense oligonucleotide which targets the 3′-untranslated region [UTR] of TS mRNA reduced TS mRNA levels, TS protein activity and cell proliferation (Ferguson P J, et al., Br J Pharmacol 1999; 127:1777-1786). This antisense oligonucleotide also increased HeLa cell sensitivity to 5-FU, 5-FUdR and raltitrexed cytotoxicity, but did not influence sensitivity to non-TS targeting drugs including cisplatin and chlorambucil (Ferguson P J, et al., 1999; ibid.). Similar results were obtained in TS-overexpressing HeLa cells selected in vitro for resistance to 5-FudR (Ferguson, et al., 2001, Br. J. Pharmacol. 134:1437-1446) and in human colon carcinoma HT-29 cells both in vitro and grown as explants in immunocompromised mice (Berg et al., 2001, J. Pharmacol. Exp. Ther. 298:477-484).

The physiological effects of targeting different regions of TS mRNA have also been investigated and indicated that certain regions of TS mRNA are not effectively targeted by AS ODNs (Ferguson P J, et al., 1999; ibid.; Berg R W, et al., Cancer Gene Ther 2003; 10:278-286) and targeting the translation start site increases TS gene transcription (DeMoor J M, et al., Exp Cell Res 1998; 243: 11-21).

A recent report also indicates that an antisense oligonucleotide that targets nucleotides 201 to 217 of the TS mRNA, inhibited the proliferation of HEK cells, but was relatively ineffective in inhibiting proliferation of HeLa cells (Lin S B, et al., Mol Pharmacol 2001; 60: 474-479). This contrasted with the higher sensitivity demonstrated by HeLa cells to inhibition with 5-FUdR. The report speculated that different levels of thymidine kinase activity might account for this observation, but did not investigate the effect of the antisense oligonucleotide on TS mRNA or protein levels in HeLa cells.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide antisense oligonucleotides targeted to the coding region of a thymidylate synthase gene and uses thereof. In accordance with an aspect of the present invention, there is provided an antisense oligonucleotide targeted to thymidylate synthase for use to inhibit the proliferation of cancer cells in a subject, said antisense oligonucleotide having a sequence between about 7 and about 50 nucleotides in length comprising 7 or more consecutive nucleotides complementary to the coding region of a human thymidylate synthase mRNA, wherein said antisense oligonucleotide inhibits the proliferation of said cancer cells without decreasing the level of thymidylate synthase mRNA in said cells.

In accordance with another aspect of the present invention, there is provided an antisense oligonucleotide targeted to thymidylate synthase for use to induce apoptosis in cancer cells in a subject, said antisense oligonucleotide having a sequence between about 7 and about 50 nucleotides in length comprising a sequence of 7 or more consecutive nucleotides complementary to the coding region of a human thymidylate synthase mRNA, wherein said antisense oligonucleotide induces apoptosis of said cancer cells without decreasing the level of thymidylate synthase mRNA in said cells.

In accordance with another aspect of the present invention, there is provided an antisense oligonucleotide having a sequence between about 7 and about 50 nucleotides in length comprising a sequence of 7 or more consecutive nucleotides complementary to the coding region of a human thymidylate synthase mRNA, wherein said antisense oligonucleotide inhibits proliferation of cancer cells without decreasing the level of human thymidylate synthase mRNA in said cells.

In accordance with another aspect of the present invention, there is provided a method of inhibiting the proliferation of cancer cells in a subject, said method comprising contacting said cells with an effective amount of an antisense oligonucleotide targeted to thymidylate synthase, said antisense oligonucleotide having a sequence between about 7 and about 50 nucleotides in length comprising a sequence of 7 or more consecutive nucleotides complementary to the coding region of a human thymidylate synthase mRNA, wherein said antisense oligonucleotide inhibits the proliferation of said cancer cells without decreasing the level of thymidylate synthase mRNA in said cells.

In accordance with another aspect of the present invention, there is provided a method of increasing apoptosis in cancer cells in a subject comprising contacting said cells with an effective amount of an antisense oligonucleotide targeted to thymidylate synthase, said antisense oligonucleotide having a sequence between about 7 and about 50 nucleotides in length comprising a sequence of 7 or more consecutive nucleotides complementary to the coding region of a human thymidylate synthase mRNA, wherein said antisense oligonucleotide inhibits the proliferation of said cancer cells without decreasing the level of thymidylate synthase mRNA in said cells.

In accordance with another aspect, there is provided a method of treating cancer in a subject in need thereof comprising administering to said subject an effective amount of an antisense oligonucleotide of the invention.

In accordance with another aspect of the present invention, there is provided a use of an antisense oligonucleotide targeted to thymidylate synthase, said antisense oligonucleotide having a sequence between about 7 and about 50 nucleotides in length comprising a sequence of 7 or more consecutive nucleotides complementary to the coding region of a human thymidylate synthase mRNA to inhibit the proliferation of cancer cells in a subject, wherein said antisense oligonucleotide inhibits the proliferation of said cancer cells without decreasing the level of thymidylate synthase mRNA in said cells.

In accordance with another aspect of the present invention, there is provided a use of an antisense oligonucleotide targeted to thymidylate synthase, said antisense oligonucleotide having a sequence between about 7 and about 50 nucleotides in length comprising a sequence of 7 or more consecutive nucleotides complementary to the coding region of a human thymidylate synthase mRNA to induce apoptosis of cancer cells in a subject, wherein said antisense oligonucleotide induces apoptosis of said cancer cells without decreasing the level of thymidylate synthase mRNA in said cells.

### BRIEF DESCRIPTION OF THE FIGURES

These and other features of the invention will become more apparent in the following detailed description in which reference is made to the appended drawings.

FIG. 1 provides the sequence of the human thymidylate synthase mRNA [SEQ ID NO:1].

FIG. 2 illustrates the effect of an antisense oligonucleotide [SEQ ID NO:2] according to one embodiment of the invention on in vitro proliferation of (A) HeLa cancer cells and (B) MCF-7 cancer cells.

FIG. 3 illustrates the effect of different doses of an antisense oligonucleotide [SEQ ID NO:2] according to one embodiment of the invention on in vitro proliferation of MCF-7 cancer cells.

FIG. 4 illustrates the effect of an antisense oligonucleotide [SEQ ID NO:2] according to one embodiment of the invention on thymidylate synthase mRNA levels in HeLa and MCF-7 cancer cells in vitro.

FIG. 5 illustrates the effect of an antisense oligonucleotide [SEQ ID NO:2] according to one embodiment of the invention on thymidylate synthase mRNA levels in HeLa and MCF-7 cancer cells in vitro.

FIG. 6 illustrates the effect of an antisense oligonucleotide [SEQ ID NO:2] according to one embodiment of the invention on thymidylate synthase protein activity in HeLa and MCF-7 cancer cells in vitro.

FIG. 7 illustrates the sensitivity of HeLa cancer cells to (A) raltitrexed, (B) 5-FudR and (C) cisplatin cytotoxicity following antisense oligonucleotide pre-treatment.

FIG. 8 illustrates the sensitivity of MCF-7 cancer cells to (A) raltitrexed, (B) 5-FudR and (C) cisplatin cytotoxicity following antisense oligonucleotide pre-treatment.

FIG. 9 depicts flow cytometric analysis of apoptosis in MCF-7 cancer cells following antisense oligonucleotide treatment.

FIG. 10 depicts flow cytometric analysis of cell cycle in HeLa cancer cells following antisense oligonucleotide treatment; (A) control oligonucleotide [SEQ ID NO:3] and (B) antisense oligonucleotide [SEQ ID NO:2].

FIG. 11 depicts flow cytometric analysis of apoptosis in MCF-7 cancer cells following antisense oligonucleotide treatment; (A) control oligonucleotide [SEQ ID NO:3] and (B) antisense oligonucleotide [SEQ ID NO:2].

FIG. 12 depicts flow cytometric analysis of cell cycle in (A) HeLa cells and (B) MCF-7 cancer cells following antisense oligonucleotide treatment.

### DETAILED DESCRIPTION OF THE INVENTION

The present invention provides for antisense oligonucleotides directed to the coding region of a mammalian thymidylate synthase mRNA that are capable of inhibiting the proliferation of cancer cells without decreasing the level of thymidylate synthase mRNA in the cells. In accordance with one aspect of the present invention, the antisense oligonucleotides are capable of inducing apoptosis in the cancer cells. The present invention thus further provides for the use of the antisense oligonucleotides to inhibit the proliferation of cancer cells and/or to induce apoptosis in cancer cells. Methods of treating cancer with the antisense oligonucleotides, alone or in combination with other therapeutics, are also provided.

A specific embodiment of the present invention relates to breast cancer. In accordance with this aspect of the invention, the antisense oligonucleotides are capable of inhibiting the proliferation of breast cancer cells without decreasing the level of thymidylate synthase mRNA in the cells. In one embodiment, the antisense oligonucleotides are capable of inducing apoptosis in breast cancer cells.

In accordance with the present invention, the antisense oligonucleotides demonstrate the above abilities in cells of at least one cancer cell type. In one aspect of the present invention, the antisense oligonucleotides of the present invention are also capable of exerting alternate antisense effects, e.g. a standard antisense effect of decreasing thymidylate synthase mRNA levels, in other cancer cell types. In these cancer cells, the antisense oligonucleotides may also act to enhance the cytotoxic effects of thymidylate synthase targeting drugs. Accordingly, the present invention further provides for the use of the antisense oligonucleotides in combination therapies with thymidylate synthase targeting drugs in the treatment of these types of cancers. The effect of the antisense oligonucleotides in different cancer cell lines can be readily determined by analysis of their effect on thymidylate synthase mRNA levels using standard techniques known in the art, representative examples of which are described herein.

Definitions

Unless defined otherwise, all technical and scientific terms used herein have the same meaning as commonly understood by one of ordinary skill in the art to which this invention pertains.

The terms “antisense oligonucleotide” and “antisense oligodeoxynucleotide” (ODN) as used herein refer to a nucleotide sequence that is complementary to a mRNA for a target gene. In the context of the present invention, the target gene is the gene encoding a mammalian thymidylate synthase protein.

The term “selectively hybridise” as used herein refers to the ability of a nucleic acid to bind detectably and specifically to a second nucleic acid. Oligonucleotides selectively hybridise to target nucleic acid strands under hybridisation and wash conditions that minimise appreciable amounts of detectable binding to non-specific nucleic acids. High stringency conditions can be used to achieve specific hybridization conditions as known in the art. Typically, hybridization and washing are performed at high stringency according to conventional hybridization procedures and employing one or more washing step in a solution comprising 1-3×SSC, 0.1-1% SDS at 50-70° C. for 5-30 minutes.

The term “corresponds to” as used herein with reference to nucleic acid sequences means a polynucleotide sequence that is identical to all or a portion of a reference polynucleotide sequence. In contradistinction, the term “complementary to” is used herein to mean that the polynucleotide sequence is identical to all or a portion of the complement of a reference polynucleotide sequence. For illustration, the nucleotide sequence “TATAC” corresponds to a reference sequence “TATAC” and is complementary to a reference sequence “GTATA”.

The following terms are used herein to describe the sequence relationships between two or more polynucleotides: “reference sequence,” “window of comparison” and “percent sequence identity.” A “reference sequence” is a defined sequence used as a basis for a sequence comparison; a reference sequence may be a subset of a larger sequence, for example, as a segment of a full-length mRNA, cDNA, gene sequence, or may comprise a complete mRNA, cDNA, gene sequence. Generally, a reference polynucleotide sequence is at least 20 nucleotides in length, and often at least 50 nucleotides in length.

A “window of comparison”, as used herein, refers to a conceptual segment of the reference sequence of at least 15 contiguous nucleotide positions over which a candidate sequence may be compared to the reference sequence and wherein the portion of the candidate sequence in the window of comparison may comprise additions or deletions (i.e. gaps) of 20 percent or less as compared to the reference sequence (which does not comprise additions or deletions) for optimal alignment of the two sequences. The present invention contemplates various lengths for the window of comparison, up to and including the full length of either the reference or candidate sequence. Optimal alignment of sequences for aligning a comparison window may be conducted using the local homology algorithm of Smith and Waterman (Adv. Appl. Math. (1981) 2:482), the homology alignment algorithm of Needleman and Wunsch (J. Mol. Biol. (1970) 48:443), the search for similarity method of Pearson and Lipman (Proc. Natl. Acad. Sci. (U.S.A.) (1988) 85:2444), using computerised implementations of these algorithms (such as GAP, BESTFIT, FASTA, and TFASTA in the Wisconsin Genetics Software Package Release 7.0, Genetics Computer Group, 573 Science Dr., Madison, Wis.), using publicly available computer software such as ALIGN or Megalign (DNASTAR), or by inspection. The best alignment (i.e. resulting in the highest percentage of identity over the comparison window) is then selected.

The term “percent (%) sequence identity,” as used herein with respect to a reference sequence is defined as the percentage of nucleotide residues in a candidate sequence that are identical with the residues in the reference sequence over the window of comparison after optimal alignment of the sequences and introducing gaps, if necessary, to achieve the maximum percent sequence identity, without considering any conservative substitutions as part of the sequence identity.

The term “inhibit,” as used herein, means to decrease, reduce, slow-down or prevent.

The term “induce,” as used herein, with reference to the effect of an antisense oligonucleotide on a physiological event, means to stimulate or increase the event. In this context, prior to induction, the event can be non-existent, in which case the induction initiates the event, or already proceeding, in which case the induction increases the level and/or rate at which the event is proceeding.

The terms “therapy” and “treatment,” as used interchangeably herein, refer to an intervention performed with the intention of improving a subject's status. The improvement can be subjective or objective and is related to ameliorating the symptoms associated with, preventing the development of, or altering the pathology of a disease or disorder being treated. Thus, the terms therapy and treatment are used in the broadest sense, and include the prevention (prophylaxis), moderation, reduction, and curing of a disease or disorder at various stages. Preventing deterioration of a subject's status is also encompassed by the term. Subjects in need of therapy/treatment thus include those already having the disease or disorder as well as those prone to, or at risk of developing, the disease or disorder and those in whom the disease or disorder is to be prevented.

The term “ameliorate” or “amelioration” includes the arrest, prevention, decrease, or improvement in one or more the symptoms, signs, and features of the disease being treated, both temporary and long-term.

The term “subject” or “patient” as used herein refers to an animal in need of treatment.

The term “animal,” as used herein, refers to both human and non-human animals, including, but not limited to, mammals, birds and fish.

Administration of the compounds of the invention “in combination with” one or more further therapeutic agents, is intended to include simultaneous (concurrent) administration and consecutive administration. Consecutive administration is intended to encompass administration of the therapeutic agent(s) and the compound(s) of the invention to the subject in various orders and via various routes.

As used herein, the term “about” refers to a +/−10% variation from the nominal value. It is to be understood that such a variation is always included in any given value provided herein, whether or not it is specifically referred to.

Antisense Oligonucleotides

Selection and Characteristics

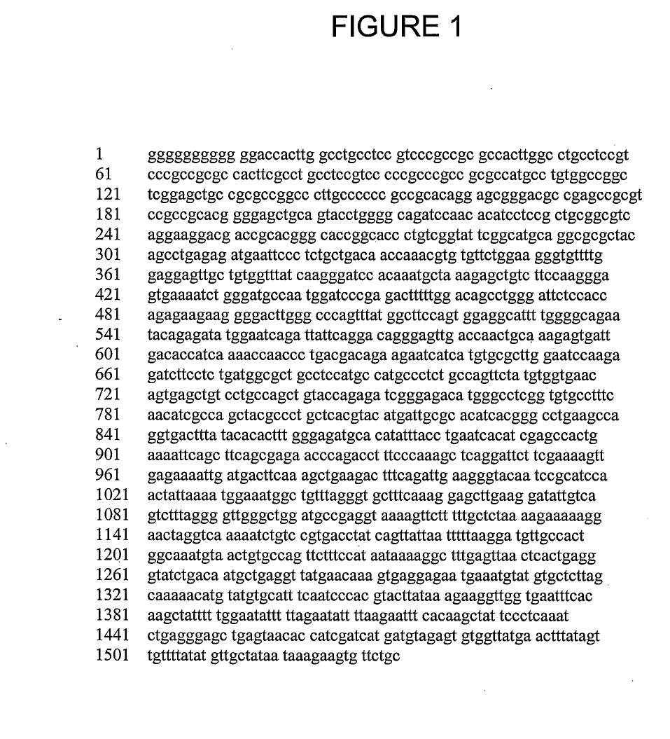

The antisense oligonucleotides of the present invention are targeted to a mammalian thymidylate synthase (TS) gene and thus comprise a nucleotide sequence complementary to a region of the mRNA transcribed from the gene. The sequences of various mammalian TS genes and mRNAs are known in the art and can be readily obtained from Genbank (maintained by the National Center for Biotechnology Information). In one embodiment of the invention, the antisense oligonucleotides are targeted to a human TS gene. In another embodiment, the antisense oligonucleotides comprise a sequence complementary to a portion of a human TS mRNA. The sequence for human TS mRNA can be accessed from GenBank under Accession No. X02308 and is provided herein as FIG. 1 [SEQ ID NO:1].

In accordance with the present invention, the antisense oligonucleotides are targeted to the coding region of the TS mRNA. In the human TS mRNA shown in FIG. 1 [SEQ ID NO:1], the coding region extends from position 106 to position 1047. In one embodiment of the present invention, the antisense oligonucleotides are targeted to the region of a human TS mRNA represented by nucleotides 106-1047 of SEQ ID NO:1.

The antisense oligonucleotides in accordance with the present invention are selected from a sequence complementary to the TS mRNA such that the sequence exhibits the least likelihood of forming duplexes, hair-pins, or of containing homooligomer/sequence repeats. The oligonucleotide may further contain a GC clamp. One skilled in the art will appreciate that these properties can be determined qualitatively using various computer modelling programs, for example, the program OLIGO® Primer Analysis Software, Version 5.0 (distributed by National Biosciences, Inc., Plymouth, Minn.).

It is understood in the art that an antisense oligonucleotide need not have 100% identity with the complement of its target sequence. The antisense oligonucleotides in accordance with the present invention have a sequence that is at least about 75% identical to the complement of the target sequence. In one embodiment of the present invention, the antisense oligonucleotides have a sequence that is at least about 90% identical to the complement of the target sequence. In a related embodiment, they have a sequence that is at least about 95% identical to the complement of the target sequence, allowing for gaps or mismatches of several bases. Identity can be determined, for example, by using the BLASTN program of the University of Wisconsin Computer Group (GCG) software or provided on the NCBI website.

In accordance with the present invention, antisense oligonucleotides are typically between about 7 and about 50 nucleotides in length and comprise a sequence of 7 or more consecutive nucleotides complementary to a portion of the coding region of a mammalian TS mRNA. In one embodiment, the antisense oligonucleotides are between about 7 and about 40 nucleotides in length. In another embodiment, the antisense oligonucleotides are between about 7 and about 35 nucleotides in length. In a further embodiment, the antisense oligonucleotides are between about 10 and about 50 nucleotides in length. In other embodiments, the antisense oligonucleotides are between about 12 and about 50 nucleotides, between about 12 and about 35 nucleotides and between about 15 and about 35 nucleotides in length.

In one embodiment of the present invention, the antisense oligonucleotide comprises a sequence complementary to a portion of the coding region of a human TS mRNA. In another embodiment, the antisense oligonucleotide comprises a sequence complementary to a portion of the coding region of a human TS mRNA between nucleotide 109 to nucleotide 500 of SEQ ID NO:1. In a further embodiment, the antisense oligonucleotide comprises a sequence complementary to a portion of the coding region of a human TS mRNA between nucleotide 109 to nucleotide 400 of SEQ ID NO: 1. In another embodiment, the antisense oligonucleotide comprises a sequence complementary to a portion of the coding region of a human TS mRNA between nucleotide 109 to nucleotide 300 of SEQ ID NO:1. In other embodiments, the antisense oligonucleotide comprises a sequence complementary to a portion of the coding region of a human TS mRNA between nucleotide 150 to nucleotide 300 of SEQ ID NO:1, to a portion of the coding region of a human TS mRNA between nucleotide 200 to nucleotide 300 of SEQ ID NO:1, to a portion of the coding region of a human TS mRNA between nucleotide 210 to nucleotide 300 of SEQ ID NO:1, to a portion of the coding region of a human TS mRNA between nucleotide 214 to nucleotide 300 of SEQ ID NO:1, and to a portion of the coding region of a human TS mRNA between nucleotide 214 to nucleotide 264 of SEQ ID NO:1.

In alternative embodiments, the antisense oligonucleotide comprises a sequence complementary to a portion of the coding region of a human TS mRNA between nucleotide 150 to ‘nucleotide 250 of SEQ ID NO:1, to a portion of the coding region of a human TS mRNA between nucleotide 150 to nucleotide 240 of SEQ ID NO:1 and to a portion of the coding region of a human TS mRNA between nucleotide 150 to nucleotide 235 of SEQ ID NO:1.

In certain embodiments of the present invention, the sequence of the antisense oligonucleotide is other than the sequence complementary to nucleotides 201-217 of SEQ ID NO:1.

In a specific embodiment of the present invention, the antisense oligonucleotide comprises 7 or more consecutive nucleotides of the sequence as set forth in SEQ ID NO:2.

| | |
|---|---|
| 5′-CAGCGGAGGATGTGTTGGAT-3′ | [SEQ ID NO: 2] |

In another embodiment, the antisense oligonucleotide comprises 10 or more consecutive nucleotides of the sequence as set forth in SEQ ID NO:2. In a further embodiment, the antisense oligonucleotide comprises12 or more consecutive nucleotides of the sequence as set forth in SEQ ID NO:2. In a further embodiment, the antisense oligonucleotide comprises15 or more consecutive nucleotides of the sequence as set forth in SEQ ID NO:2.

In another embodiment, the antisense oligonucleotide comprises the sequence as set forth in SEQ NO:2.

The term “antisense oligonucleotides” as used herein includes other oligomeric antisense compounds, including oligonucleotide mimetics, modified oligonucleotides, and chimeric antisense compounds. Chimeric antisense compounds are antisense compounds that contain two or more chemically distinct regions, each made up of at least one monomer unit.

Thus, in the context of this invention, the term “oligonucleotide” refers to an oligomer or polymer of ribonucleic acid (RNA), deoxyribonucleic acid (DNA), or RNA or DNA mimetics. This term, therefore, includes oligonucleotides composed of naturally-occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

As is known in the art, a nucleoside is a base-sugar combination and a nucleotide is a nucleoside that further includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Specific examples of antisense compounds useful in this invention include oligonucleotides containing modified backbones or non-natural internucleoside linkages. As defined in this specification, oligonucleotides having modified backbones include both those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

In one embodiment of the present invention, the antisense oligonucleotide comprises one or more phosphorothioate linkage. In another embodiment, the antisense oligonucleotide comprises a region that comprises phosphorothioate internucleotide linkages. In another embodiment, the region comprises four, five or six nucleotides of the oligonucleotide. In a further embodiment, the antisense oligonucleotide comprises phosphorothioate internucleotide linkages that link all the nucleotides of the oligonucleotide.

Exemplary modified oligonucleotide backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include morpholino linkages (formed in part from the sugar portion of a nucleoside); siloxane backbones; sulfide, sulfoxide and sulphone backbones; formacetyl and thioformacetyl backbones; methylene formacetyl and thioformacetyl backbones; alkene containing backbones; sulphamate backbones; methyleneimino and methylenehydrazino backbones; sulphonate and sulfonamide backbones; amide backbones; and others having mixed N, O, S and CH₂ component parts.

The present invention also contemplates oligonucleotide mimetics in which both the sugar and the internucleoside linkage of the nucleotide units are replaced with novel groups. The base units are maintained for hybridisation with an appropriate nucleic acid target compound. An example of such an oligonucleotide mimetic, which has been shown to have excellent hybridisation properties, is a peptide nucleic acid (PNA) [Nielsen et al., Science, 254:1497-1500 (1991)]. In PNA compounds, the sugar-backbone of an oligonucleotide is replaced with an amide containing backbone, in particular an aminoethylglycine backbone. The nucleobases are retained and are bound directly or indirectly to aza-nitrogen atoms of the amide portion of the backbone.

The present invention also contemplates oligonucleotides comprising “locked nucleic acids” (LNAs), which are novel conformationally restricted oligonucleotide analogues containing a methylene bridge that connects the 2′-O of ribose with the 4′-C (see, Singh et al., Chem. Commun., 1998, 4:455-456). LNA and LNA analogues display very high duplex thermal stabilities with complementary DNA and RNA, stability towards 3′-exonuclease degradation, and good solubility properties. Synthesis of the LNA analogues of adenine, cytosine, guanine, 5-methylcytosine, thymine and uracil, their oligomerization, and nucleic acid recognition properties have been described (see Koshkin et al., Tetrahedron, 1998, 54:3607-3630). Studies of mis-matched sequences show that LNA obey the Watson-Crick base pairing rules with generally improved selectivity compared to the corresponding unmodified reference strands.

Antisense oligonucleotides containing LNAs have been described (Wahlestedt et al., Proc. Natl. Acad. Sci. U.S.A., 2000, 97:5633-5638), which were efficacious and non-toxic. In addition, the LNA/DNA copolymers were not degraded readily in blood serum and cell extracts.

LNAs form duplexes with complementary DNA or RNA or with complementary LNA, with high thermal affinities. The universality of LNA-mediated hybridization has been emphasized by the formation of exceedingly stable LNA:LNA duplexes (Koshkin et al., J. Am. Chem. Soc., 1998, 120:13252-13253). LNA:LNA hybridization was shown to be the most thermally stable nucleic acid type duplex system, and the RNA-mimicking character of LNA was established at the duplex level. Introduction of three LNA monomers (T or A) resulted in significantly increased melting points toward DNA complements.

Synthesis of 2′-amino-LNA (Singh et al., J. Org. Chem., 1998, 63, 10035-10039) and 2′-methylamino-LNA has been described and thermal stability of their duplexes with complementary RNA and DNA strands reported. Preparation of phosphorothioate-LNA and 2′-thio-LNA have also been described (Kumar et al., Bioorg. Med. Chem. Lett., 1998, 8:2219-2222).

Modified oligonucleotides may also contain one or more substituted sugar moieties. For example, oligonucleotides may comprise sugars with one of the following substituents at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Examples of such groups are: O[(CH₂)ₙ O]ₘ CH₃, O(CH₂)ₙ OCH₃, O(CH₂)ₙ NH₂, O(CH₂)ₙ CH₃, O(CH₂)ₙ ONH₂, and O(CH₂)ₙ ON[(CH₂)ₙ CH₃)]₂, where n and m are from 1 to about 10. Alternatively, the oligonucleotides may comprise one of the following substituents at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Specific examples include 2′-methoxyethoxy (2′-O—CH₂ CH₂ OCH₃, also known as 2′-O-(2-methoxyethyl) or 2′-MOE) [Martin et al., Helv. Chim. Acta, 78:486-504(1995)], 2′-dimethylaminooxyethoxy (O(CH₂)₂ ON(CH₃)₂ group, also known as 2′-DMAOE), 2′-methoxy (2′-O—CH₃), 2′-aminopropoxy (2′-OCH₂ CH₂ CH₂NH₂) and 2′-fluoro (2′-F).

In one embodiment of the present invention, the antisense oligonucleotide comprises at least one nucleotide comprising a substituted sugar moiety. In another embodiment, the antisense oligonucleotide comprises at least one 2′-O-(2-methoxyethyl) or 2′-MOE modified nucleotide.

Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include modifications or substitution's to the nucleobase. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil(pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. I., ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi, Y. S., (1993) Antisense Research and Applications, pp 276-278, Crooke, S. T. and Lebleu, B., ed., CRC Press, Boca Raton].

Another oligonucleotide modification included in the present invention is the chemically linkage to the oligonucleotide of one or more moieties or conjugates which enhance the activity, cellular distribution or cellular uptake of the oligonucleotide. Such moieties include, but are not limited to, lipid moieties such as a cholesterol moiety [Letsinger et al., Proc. Natl. Acad. Sci. USA, 86:6553-6556 (1989)], cholic acid [Manoharan et al., Bioorg. Med. Chem. Let., 4:1053-1060 (1994)], a thioether, e.g. hexyl-S-tritylthiol [Manoharan et al., Ann. N.Y. Acad. Sci., 660:306-309 (1992); Manoharan et al., Bioorg. Med. Chem. Lett., 3:2765-2770 (1993)], a thiocholesterol [Oberhauser et al., Nucl. Acids Res., 20:533-538 (1992)], an aliphatic chain, e.g. dodecandiol or undecyl residues [Saison-Behmoaras et al., EMBO J., 10:1111-1118 (1991); Kabanov et al., FEBS Lett., 259:327-330 (1990); Svinarchuk et al., Biochimie, 75:49-54 (1993)], a phospholipid, e.g. di-hexadecyl-rac-glycerol or triethylammonium 1,2-di-O-hexadecyl-rac-glycero-3-H-phosphonate [Manoharan et al., Tetrahedron Lett., 36:3651-3654 (1995); Shea et al., Nucl. Acids Res., 18:3777-3783 (1990)], a polyamine or a polyethylene glycol chain [Manoharan et al., Nucleosides & Nucleotides, 14:969-973 (1995)], or adamantane acetic acid [Manoharan et al., Tetrahedron Lett., 36:3651-3654 (1995)], a palmityl moiety [Mishra et al., Biochim. Biophys. Acta, 1264:229-237 (1995)], or an octadecylamine or hexylamino-carbonyl-oxycholesterol moiety [Crooke et al., J. Pharmacol. Exp. Ther., 277:923-937 (1996)].

One skilled in the art will recognise that it is not necessary for all positions in a given oligonucleotide to be uniformly modified. The present invention, therefore, contemplates the incorporation of more than one of the aforementioned modifications into a single oligonucleotide or even at a single nucleoside within the oligonucleotide. The present invention thus further includes antisense compounds that are chimeric compounds. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease that cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridising to the same target region. Cleavage of the RNA target can be routinely detected by gel electrophoresis and, if necessary, associated nucleic acid hybridisation techniques known in the art.

In one embodiment of the present invention, the antisense oligonucleotides comprise a phosphorothioate backbone in combination with at least one 2′-MOE modified sugar. In another embodiment, the antisense oligonucleotides comprise a phosphorothioate backbone in combination with one or more 2′-MOE modified sugars at the 3′ and 5′ ends of the oligonucleotide.

In the context of the present invention, an antisense oligonucleotide is “nuclease resistant” when it has either been modified such that it is not susceptible to degradation by DNA and RNA nucleases or alternatively has been placed in a delivery vehicle which in itself protects the oligonucleotide from DNA or RNA nucleases. Nuclease resistant oligonucleotides include, for example, methyl phosphonates, phosphorothioates, phosphorodithioates, phosphotriesters, and morpholino oligomers. Suitable delivery vehicles for conferring nuclease resistance include, for example, liposomes. In one embodiment of the present invention, the antisense oligonucleotides are nuclease resistant.

The present invention further contemplates antisense oligonucleotides that contain groups for improving the pharmacokinetic properties of the oligonucleotide, or groups for improving the pharmacodynamic properties of the oligonucleotide.

Short Interfering RNA (siRNA) Molecules

The present invention further contemplates that the antisense oligonucleotides may be in the form of siRNA molecules. The siRNA molecule can be double stranded (i.e. a dsRNA molecule comprising an antisense strand and a complementary sense strand) or single-stranded (i.e. a ssRNA molecule comprising just an antisense strand). The siRNA molecules can comprise a duplex, asymmetric duplex, hairpin or asymmetric hairpin secondary structure, having self-complementary sense and antisense strands.

The siRNA molecule can be a double-stranded RNA (dsRNA) comprising two separate complementary RNA strands. The RNA strands of the dsRNA may be the same length in nucleotides, or may be different in length. In one embodiment, the siRNA is a dsRNA. In another embodiment, the siRNA is a dsRNA wherein both RNA strands are the same length.

The dsRNA molecules of the present invention also include siRNA molecules assembled from a single oligonucleotide in a stem-loop structure, wherein self-complementary sense and antisense regions of the siRNA molecule are linked by means of a nucleic acid based or non-nucleic acid-based linker(s), as well as circular single-stranded RNA having two or more loop structures and a stem comprising self-complementary sense and antisense strands, wherein the circular RNA can be processed either in vivo or in vitro to generate an active siRNA molecule capable of mediating RNAi.

Small hairpin RNA (shRNA) molecules thus are also contemplated by the present invention. These molecules comprise a specific antisense sequence in addition to the reverse complement (sense) sequence, typically separated by a spacer or loop sequence. Cleavage of the spacer or loop provides a single-stranded RNA molecule and its reverse complement, such that they may anneal to form (optionally with additional processing steps that may result in addition or removal of one, two, three or more nucleotides from the 3′ end and/or the 5′ end of either or both strands) a dsRNA molecule. The spacer can be of a sufficient length to permit the antisense and sense sequences to anneal and form a double-stranded structure (or stem) prior to cleavage of the spacer (and, optionally, subsequent processing steps that may result in addition or removal of one, two, three, four, or more nucleotides from the 3′ end and/or the 5′ end of either or both strands). The spacer sequence is typically an unrelated nucleotide sequence that is situated between two complementary nucleotide sequence regions which, when annealed into a double-stranded nucleic acid, comprise a shRNA (see, for example, Brummelkamp et al., 2002 Science 296:550; Paddison et al., 2002 Genes Develop. 16:948; Paul et al., Nat. Biotechnol. 20:505-508 (2002); Grabarek et al., BioTechniques 34:734-44 (2003)). The spacer sequence generally comprises between about 3 and about 100 nucleotides.

The ssRNA molecules according to the present invention are generally single-stranded RNA molecules with little or no secondary structure.

The overall length of the siRNA molecules of the present invention can vary from about 14 to about 200 nucleotides depending on the type of siRNA molecule being designed. For example, when the siRNA molecule is a dsRNA or ssRNA molecule, the length can vary from about 14 to about 50 nucleotides, whereas when the siRNA is a shRNA or circular molecule, the length can vary from about 40 nucleotides to about 200 nucleotides. In one embodiment of the present invention, the siRNA molecule is a dsRNA or ssRNA molecule between about 17 and about 30 nucleotides in length. In another embodiment, the siRNA molecule is a dsRNA or ssRNA molecule between about 19 and about 25 nucleotides in length. In another embodiment, the siRNA molecule is a dsRNA or ssRNA molecule between about 21 to about 23 nucleotides in length. In an alternative embodiment, the siRNA molecule is a shRNA molecule or circular siRNA molecule between about 50 and about 100 nucleotides in length. In a further embodiment, the siRNA molecule is a shRNA molecule between about 50 to about 60 nucleotides in length.

As indicated above, the siRNA molecule comprises an antisense strand that includes a specific antisense sequence complementary to all or a portion of a target mRNA sequence. One skilled in the art will appreciate that the entire length of the antisense strand comprised by the siRNA molecule does not need to be complementary to the target sequence. Thus, the antisense strand of the siRNA molecules may comprise nucleotide sequences at the 5′ and/or 3′ termini that are not complementary to the target sequence. Such non-complementary nucleotides may provide additional functionality to the siRNA molecule. For example, they may provide a restriction enzyme recognition sequence or a “tag” that facilitates detection, isolation or purification. Alternatively, the additional nucleotides may provide a self-complementary sequence that allows the siRNA to adopt a hairpin configuration. Such configurations are useful when the siRNA molecule is a shRNA molecule, as described above.

Accordingly, within its overall length of about 14 to about 200 nucleotides, the siRNA molecules of the present invention comprise a specific antisense sequence of between about 14 to about 50 nucleotides in length that is complementary to all or a portion of a selected target mRNA sequence. In one embodiment, the length of the specific antisense sequence is from about 14 to about 30 nucleotides. In another embodiment, the length of the specific antisense sequence is from about 19 to about 25 nucleotides. In a further embodiment, the length of the specific antisense sequence is from about 19 to about 23 nucleotides. In another embodiment, the length of the specific antisense sequence is from about 21 to about 23 nucleotides.

The specific antisense sequence of the siRNA molecules of the present invention may exhibit variability by differing (e.g. by nucleotide substitution, including transition or transversion) at one, two, three, four or more nucleotides from the sequence of the target mRNA. When such nucleotide substitutions are present in the antisense strand of a dsRNA molecule, the complementary nucleotide in the sense strand with which the substitute nucleotide would typically form hydrogen bond base-pairing may or may not be correspondingly substituted. dsRNA molecules in which one or more nucleotide substitution occurs in the sense sequence, but not in the antisense strand, are also contemplated by the present invention. When the antisense sequence of an siRNA molecule comprises one or more mismatches between the nucleotide sequence of the siRNA and the target nucleotide sequence, as described above, the mismatches may be found at the 3′ terminus, the 5′ terminus or in the central portion of the antisense sequence.

According to the present invention, siRNA molecules having a duplex or double-stranded structure, for example dsRNA or shRNA, can have blunt ends, or can have 3′ and/or 5′ overhangs. As used herein, “overhang” refers to the unpaired nucleotide or nucleotides that protrude from a duplex structure when a 3′-terminus of one RNA strand extends beyond the 5′-terminus of the other strand (3′ overhang), or vice versa (5′ overhang). The nucleotides comprising the overhang can be ribonucleotides, deoxyribonucleotides or modified versions thereof. In one embodiment, at least one strand of the siRNA molecule has a 3′ overhang from about 1 to about 6 nucleotides in length. In other embodiments, the 3′ overhang is from about 1 to about 5 nucleotides, from about 1 to about 3 nucleotides and from about 2 to about 4 nucleotides in length.

When the siRNA molecule comprises a 3′ overhang at one end of the molecule, the other end can be blunt-ended or have also an overhang (5′ or 3′). When the siRNA molecule comprises an overhang at both ends of the molecule, the length of the overhangs may be the same or different. In one embodiment, the siRNA molecule of the present invention comprises 3′ overhangs of about 1 to about 3 nucleotides on both ends of the molecule. In a further embodiment, the siRNA molecule is a dsRNA having a 3′ overhang of 2 nucleotides at both ends of the molecule. In yet another embodiment, the nucleotides comprising the overhang of the siRNA are TT dinucleotides or UU dinucleotides.

Preparation of the Antisense Oligonucleotides

The antisense oligonucleotides of the present invention can be prepared by conventional techniques well-known to those skilled in the art. For example, the oligonucleotides can be prepared using solid-phase synthesis using commercially available equipment, such as the equipment available from Applied Biosystems Canada Inc., Mississauga, Canada. As is well-known in the art, modified oligonucleotides, such as phosphorothioates and alkylated derivatives, can also be readily prepared by similar methods.

The siRNA molecules of the present invention can be prepared using several methods known in the art, such as chemical synthesis, in vitro transcription and the use siRNA expression vectors. In addition, kits providing a rapid and efficient means of constructing siRNA molecules by in vitro transcription are commercially available, for example, from Ambion (Austin, Tex.) and New England Biolabs (Beverly, Mass.) and are suitable for constructing the siRNA molecules of the present invention.

Alternatively, the antisense oligonucleotides of the present invention can be prepared by enzymatic digestion of the naturally occurring thymidylate synthase gene by methods known in the art.

Antisense oligonucleotides can also be prepared through the use of recombinant methods in which expression vectors comprising nucleic acid sequences that encode the antisense oligonucleotides are expressed in a suitable host cell. Such expression vectors can be readily constructed using procedures known in the art. Examples of suitable vectors include, but are not limited to, plasmids, phagemids, cosmids, bacteriophages, baculoviruses and retroviruses, and DNA viruses. One skilled in the art will understand that selection of the appropriate host cell for expression of the antisense oligonucleotide will be dependent upon the vector chosen. Examples of host cells include, but are not limited to, bacterial, yeast, insect, plant and mammalian cells.

One skilled in the art will also understand that the expression vector may further include regulatory elements, such as transcriptional elements, required for efficient transcription of the antisense oligonucleotide sequences. Examples of regulatory elements that can be incorporated into the vector include, but are not limited to, promoters, enhancers, terminators, and polyadenylation signals. One skilled in the art will appreciate that selection of suitable regulatory elements is dependent on the host cell chosen for expression of the antisense oligonucleotide and that such regulatory elements may be derived from a variety of sources, including bacterial, fungal, viral, mammalian or insect genes.

In accordance with the present invention, the expression vectors can be introduced into a suitable host cell or tissue by one of a variety of methods known in the art. Such methods can be found generally described in Ausubel et al. (Current Protocols in Molecular Biology, 1993 & updates, John Wiley & Sons, Inc., Boston, Mass.) and Sambrook et al. (Molecular Cloning, Third Ed., 2001, Cold Spring Harbor

Laboratory, Plainview, N.Y.) and include, for example, stable or transient transfection, lipofection, electroporation, and infection with recombinant viral vectors.

Properties and Efficacy of the Antisense Oligonucleotides

The properties and efficacy of the antisense oligonucleotides can be assessed using standard techniques. As indicated above, the antisense oligonucleotides are characterised by their ability to inhibit the growth of cancer cells without decreasing the level of thymidylate synthase (TS) mRNA in the cells. In accordance with the present invention, the antisense oligonucleotides demonstrate this ability in cells of at least one cancer cell type. In one embodiment, the antisense oligonucleotides are capable of inducing apoptosis in this cancer cell type. In a specific embodiment, the cancer cells are breast cancer cells. Thus, the antisense oligonucleotides are assessed for, and selected based on, their effect on TS mRNA levels and cell proliferation in one or more cancer cell type.

The antisense oligonucleotides of the present invention may also be capable of exerting alternate antisense effects e.g. a standard antisense effect of decreasing thymidylate synthase mRNA levels, in other cancer cell types. The effect of the antisense oligonucleotides in different cancer cell lines can be readily determined using standard techniques known in the art, representative examples of which are described below and in the Examples.

1. Effect on TS mRNA Levels in Cancer Cells

The effect of the antisense oligonucleotides on TS mRNA levels in cancer cells can be determined, for example, by culturing cells of a selected cancer cell line in a suitable medium. After an appropriate incubation time, the cells are transfected with the antisense oligonucleotide, for example in the presence of a commercial lipid carrier such as lipofectamine, and the incubation is continued. After this incubation, mRNA levels can be measured, for example, using Northern blot analysis or by employing RT-PCR procedures. The levels of TS mRNA in the treated cells can then be compared to an appropriate control, such as untreated cells and/or cells treated with a compound known to inhibit or induce TS expression.

Antisense oligonucleotides that have little or no effect on the level of TS mRNA in at least one cancer cell line are selected and evaluated for their ability to inhibit proliferation and/or induce apoptosis of this cancer cell line. In accordance with the present invention, “little or no” effect means that the TS mRNA levels in the treated cells are within (±) 20% of the mRNA levels in control cells, e.g. cells treated with a control oligonucleotide or untreated cells. In one embodiment, the TS mRNA levels in the treated cells are within (±) 20% of the mRNA levels in control cells treated with a control oligonucleotide. In another embodiment, the TS mRNA levels in the treated cells are within (±) 15% of the mRNA levels in control cells In a further embodiment, the TS mRNA levels in the treated cells are within (±) 10% of the mRNA levels in control cells.

The antisense oligonucleotides can be tested in one of a variety of cell lines, such as those commercially available from the American Type Culture Collection (ATCC; Manassas, Va.). In one embodiment of the present invention, in vitro testing of the antisense oligonucleotides is conducted in a human cancer cell-line. Examples of suitable cancer cell-lines for in vitro testing include, but are not limited to, breast cancer cell-lines MCF-7 and MDA-MB-231, mesothelial cell lines MSTO-211H, NCI-H-12052 and NCI-H28, ovarian cancer cell-lines OV90 and SK-OV-3, colon cancer cell-lines CaCo, HCT116 and HT29, cervical cancer cell-line HeLa, non-small cell lung carcinoma cell-lines A549 and H1299, pancreatic cancer cell-lines MIA-PaCa-2 and AsPC-1, prostatic cancer-cell line PC-3, bladder cancer cell-line T24, liver cancer cell-lineHepG2, brain cancer cell-line U-87 MG, melanoma cell-line A2058, lung cancer cell-line NCI-H460. Other examples of suitable cell-lines are known in the art.

2. Inhibition of Cancer Cell Proliferation—In Vitro Testing

The ability of the antisense oligonucleotides to inhibit proliferation of cells from a selected cancer cell line can be determined initially in vitro.

For example, inhibition of cancer cell proliferation can be assessed by culturing cells of a cancer cell line of interest in a suitable medium. After an appropriate incubation time, the cells can be transfected with the antisense oligonucleotide, for example in the presence of a commercial lipid carrier such as lipofectamine, and incubated for a further period of time. Cells are then counted and compared to an appropriate control. Suitable controls include, for example, cells treated with a control oligonucleotide (such as a scrambled form of the test oligonucleotide), cells treated with a standard chemotherapeutic and/or untreated cells.

Alternatively, the antisense oligonucleotides can be tested in vitro by determining their ability to inhibit anchorage-independent growth of tumour cells. Anchorage-independent growth is known in the art to be a good indicator of tumourigenicity. In general, anchorage-independent growth is assessed by plating cells from a selected cancer cell-line onto soft agar and determining the number of colonies formed after an appropriate incubation period. Growth of cells treated with the antisense oligonucleotide can then be compared with that of control cells (as described above).

If necessary, the toxicity of the antisense oligonucleotides can also be initially assessed in vitro using standard techniques. For example, human primary fibroblasts can be transfected in vitro with the oligonucleotide and then tested at different time points following treatment for their viability using a standard viability assay, such as the trypan-blue exclusion assay. Cells can also be assayed for their ability to synthesize DNA, for example, using a thymidine incorporation assay, and for changes in cell cycle dynamics, for example, using a standard cell sorting assay in conjunction with a fluorocytometer cell sorter (FACS).

3. Induction of Apoptosis in Cancer Cells—In Vitro Testing

The ability of the antisense oligonucleotides to induce apoptosis in the selected cancer cell line can be determined using standard techniques (see, for example, Bonifacino et al., Current Protocols in Cell Biology, J. Wiley & Sons, Inc., New York, N.Y.). For example, morphological assays can be employed, such as trypan blue exclusion, differential staining, and Hoechst staining. Alternatively, chromatin cleavage can be detected by TUNEL assays using whole cells or paraffin sections, DNA fragmentation assays using whole cells, assays of total genomic DNA, analysis of DNA fragmentation by agarose gel electrophoresis, phenol extraction of DNA for analysis of fragmentation, and detection of DNA fragmentation by pulsed-field gel electrophoresis.

Flow cytometry can also be used to assess apoptosis, for example, gross changes in cell morphology and chromatin condensation, which occur during apoptosis, can be detected by analysis with laser light beam scattering. Early events of apoptosis, dissipation of the mitochondrial transmembrane potential and caspase activation, can be detected using, for example, fluorochrome reporter groups or appropriate antibodies. Exposure of phosphatidylserine on the exterior surface of the plasma membrane can be detected by the binding of fluoresceinated annexin V. DNA fragmentation can be detected by fractional (“sub-G1”) or DNA strand-break labelling, TUNEL or In Situ End Labeling (ISEL).

4. Inhibition of Cancer Cell Proliferation—In Vivo Testing

The ability of the antisense oligonucleotides to inhibit tumour growth or proliferation in vivo can be determined in an appropriate animal model using standard techniques known in the art (see, for example, Enna, et al., Current Protocols in Pharmacology, J. Wiley & Sons, Inc., New York, N.Y.).

In general, current animal models for screening anti-tumour compounds are xenograft models, in which a human tumour has been implanted into an animal. Examples of xenograft models of human cancer include, but are not limited to, human solid tumour xenografts in mice, implanted by sub-cutaneous injection and used in tumour growth assays; human solid tumour orthotopic xenografts, implanted directly into the relevant tissue and used in tumour growth assays; human solid tumour isografts in mice, implanted by fat pad injection and used in tumour growth assays; experimental models of lymphoma and leukaemia in mice, used in survival assays, and experimental models of lung metastasis in mice.

For example, the antisense oligonucleotides can be tested in vivo on solid tumours using mice that are subcutaneously grafted bilaterally with 30 to 60 mg of a tumour fragment, or implanted with an appropriate number of cancer cells, on day 0. The animals bearing tumours are mixed before being subjected to the various treatments and controls. In the case of treatment of advanced tumours, tumours are allowed to develop to the desired size, animals having insufficiently developed tumours being eliminated. The selected animals are distributed at random to undergo the treatments and controls. Animals not bearing tumours may also be subjected to the same treatments as the tumour-bearing animals in order to be able to dissociate the toxic effect from the specific effect on the tumour. Chemotherapy generally begins from 3 to 22 days after grafting, depending on the type of tumour, and the animals are observed every day. The antisense oligonucleotide of the present invention can be administered to the animals, for example, by i.p. injection or bolus infusion. The different animal groups are weighed about 3 or 4 times a week until the maximum weight loss is attained, after which the groups are weighed at least once a week until the end of the trial.

The tumours are measured after a pre-determined time period, or they can be monitored continuously by measuring about 2 or 3 times a week until the tumour reaches a pre-determined size and/or weight, or until the animal dies if this occurs before the tumour reaches the pre-determined size/weight. The animals are then sacrificed and the tissue histology, size and/or proliferation of the tumour assessed.

Orthotopic xenograft models are an alternative to subcutaneous models and may more accurately reflect the cancer development process. In this model, tumour cells are implanted at the site of the organ of origin and develop internally. Daily evaluation of the size of the tumours is thus more difficult than in a subcutaneous model. A recently developed technique using green fluorescent protein (GFP), expressing tumours in non-invasive whole-body imaging can help to address this issue (Yang and al, Proc. Nat. Aca. Sci, (2000), pp 1206-1211). This technique utilises human or murine tumours that stably express very high levels of the Aqueora vitoria green fluorescent protein. The GFP expressing tumours can be visualised by means of externally placed video detectors, allowing for monitoring of details of tumour growth, angiogenesis and metastatic spread. Angiogenesis can be measured over time by monitoring the blood vessel density within the tumour(s). The use of this model thus allows for simultaneous monitoring of several features associated with tumour progression and has high preclinical and clinical relevance.

For the study of the effect of the antisense oligonucleotides on leukaemias, the animals are grafted with a particular number of cells, and the anti-tumour activity is determined by the increase in the survival time of the treated mice relative to the controls.

To study the effect of the antisense oligonucleotides of the present invention on tumour metastasis, tumour cells are typically treated with the composition ex vivo and then injected into a suitable test animal. The spread of the tumour cells from the site of injection is then monitored over a suitable period of time by standard techniques.

Similar methods can be employed to test the efficacy of the antisense oligonucleotides in combination with chemotherapeutic(s). Suitable controls in this case could include animals treated with the antisense oligonucleotide alone and animals treated with the chemotherapeutic(s) alone.

In vivo toxic effects of the oligonucleotides can be evaluated by measuring their effect on animal body weight during treatment and by performing haematological profiles and liver enzyme analysis after the animal has been sacrificed.

TABLE 1
Examples of xenograft models of human cancer

| Cancer Model | Cell Type |
|---|---|
| Tumour Growth Assay | Mesothelioma (NCI-H2052) |
| Human solid tumour xenografts | Prostate (PC-3, DU145) |
| in mice (sub-cutaneous injection) | Breast (MDA-MB-231, MVB-9) |
| | Colon (HT-29) |
| | Lung (NCI-H460, NCI-H209) |
| | Pancreatic (ASPC-1, SU86.86) |
| | Pancreatic: drug resistant (BxPC-3) |
| | Skin (A2058, C8161) |
| | Cervical (SIHA, HeLa-S3) |
| | Cervical: drug resistant (HeLa |
| | S3-HU-resistance) |
| | Liver (HepG2) |
| | Brain (U87-MG) |
| | Renal (Caki-1, A498) |
| | Ovary (SK-OV-3) |
| Tumour Growth Assay | Breast: drug resistant (MDA- |
| Human solid tumour isografts in | CDDP-S4, MDA-MB435-To.1) |
| mice (fat pad injection) | |
| Survival Assay | Human: Burkitts lymphoma (Non- |
| Experimental model of | Hodgkin's) (raji) |
| lymphoma and leukaemia in mice | Murine: erythroleukemia (CB7 Friend |
| | retrovirus-induced) |
| Experimental model of lung | Human: melanoma (C8161) |
| metastasis in mice | Murine: fibrosarcoma (R3) |

Pharmaceutical Compositions

The antisense oligonucleotide may be administered as a pharmaceutical composition with an appropriate pharmaceutically physiologically acceptable carrier, diluent, excipient or vehicle.

For the treatment of most types of cancer, the pharmaceutical compositions are formulated for systemic administration. For the treatment of mesothelioma, the pharmaceutical compositions can be formulated for intracavitary adminstration. The term intracavitary includes intraperitoneal, intrapericardial and intrapleural. The pharmaceutical compositions of the present invention may also be formulated for administration orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

Aqueous suspensions contain the active compound in admixture with suitable excipients including, for example, suspending agents, such as sodium carboxymethylcellulose, methyl cellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

The pharmaceutical compositions may be in the form of a sterile injectable aqueous or oleaginous suspension. This suspension may be formulated according to known art using suitable dispersing or wetting agents and suspending agents such as those mentioned above. The sterile injectable preparation may also be sterile injectable solution or suspension in a non-toxic parentally acceptable diluent or solvent, for example, as a solution in 1,3-butanediol. Acceptable vehicles and solvents that may be employed include, but are not limited to, water, Ringer's solution, lactated Ringer's solution and isotonic sodium chloride solution. Other examples are, sterile, fixed oils which are conventionally employed as a solvent or suspending medium, and a variety of bland fixed oils including, for example, synthetic mono- or diglycerides. In addition, fatty acids such as oleic acid find use in the preparation of injectables.

The pharmaceutical compositions may be in a form suitable for oral use, for example, as tablets, troches, lozenges, aqueous or oily suspensions, dispersible powders or granules, emulsion hard or soft capsules, or syrups or elixirs. Compositions intended for oral use may be prepared according to methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase may be a vegetable oil, for example, olive oil or arachis oil, or a mineral oil, for example, liquid paraffin, or it may be a mixtures of these oils. Suitable emulsifying agents may be naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

Other pharmaceutical compositions and methods of preparing pharmaceutical compositions are known in the art and are described, for example, in “Remington: The Science and Practice of Pharmacy,” Gennaro, A., Lippincott, Williams & Wilkins, Philidelphia, Pa. (2000) (formerly “Remingtons Pharmaceutical Sciences”).

Use of the Antisense Oligonucleotides

The antisense oligonucleotides of the present invention can be used in the treatment of a variety of different types of cancer, as determined by pre-clinical in vitro and in vivo studies, such as those as described above. In this context, the antisense oligonucleotides may exert either a cytotoxic or cytostatic effect resulting in a reduction in the size of a tumour, the slowing or prevention of an increase in the size of a tumour, an increase in the disease-free survival time between the disappearance or removal of a tumour and its reappearance, prevention of an initial or subsequent occurrence of a tumour (e.g. metastasis), an increase in the time to progression, reduction of one or more adverse symptom associated with a cancer, or an increase in the overall survival time of a subject having cancer.

In accordance with one aspect of the present invention, the antisense oligonucleotides are used in the treatment of breast cancer. Exemplary types of breast cancer that may be treated with the antisense oligonucleotides of the present invention include, but are not limited to, ductal carcinoma in situ (DCIS; also known as intraductal carcinoma); infiltrating (or invasive) ductal carcinoma (IDC); infiltrating (or invasive) lobular carcinoma (ILC); inflammatory breast cancer; medullary carcinoma; mucinous carcinoma; Paget's disease; malignant Phyllodes tumour (cystosarcoma phyllodes) and tubular carcinoma. Although not a true cancer, lobular carcinoma in situ (LCIS; also called lobular neoplasia) is sometimes classified as a type of noninvasive breast cancer, and may be treated with the antisense oligonucleotides of the present invention.

Examples of other cancers which may be may be treated or stabilized in accordance with the present invention include, but are not limited to, haematologic neoplasms, including leukaemias, myelomas and lymphomas; other carcinomas, including adenocarcinomas and squamous cell carcinomas; melanomas and sarcomas. Carcinomas and sarcomas are also frequently referred to as “solid tumours,” examples of commonly occurring solid tumours include, but are not limited to, cancer of the brain, breast, cervix, colon, head and neck, kidney, lung, ovary, pancreas, prostate, stomach and uterus, non-small cell lung cancer and colorectal cancer. Various forms of lymphoma also may result in the formation of a solid tumour and, therefore, are also often considered to be solid tumours.

In one embodiment of the present invention, the antisense oligonucleotides are used in the treatment of a solid tumour. In another embodiment, the antisense oligonucleotides are used in the treatment of a solid tumour selected from the group of: brain cancer, breast cancer, cervix cancer, colon cancer, head and neck cancer, kidney cancer, lung cancer, ovary cancer, pancreatic cancer, prostate cancer, stomach cancer, uterine cancer, non-small cell lung cancer and colorectal cancer.

The term “leukaemia” refers broadly to progressive, malignant diseases of the blood-forming organs. Leukaemia is typically characterized by a distorted proliferation and development of leukocytes and their precursors in the blood and bone marrow but can also refer to malignant diseases of other blood cells such as erythroleukaemia, which affects immature red blood cells. Leukaemia is generally clinically classified on the basis of (1) the duration and character of the disease—acute or chronic; (2) the type of cell involved—myeloid (myelogenous), lymphoid (lymphogenous) or monocytic, and (3) the increase or non-increase in the number of abnormal cells in the blood—leukaemic or aleukaemic (subleukaemic). Leukaemia includes, for example, acute nonlymphocytic leukaemia, chronic lymphocytic leukaemia, acute granulocytic leukaemia, chronic granulocytic leukaemia, acute promyelocytic leukaemia, adult T-cell leukaemia, aleukaemic leukaemia, aleukocythemic leukaemia, basophylic leukaemia, blast cell leukaemia, bovine leukaemia, chronic myelocytic leukaemia, leukaemia cutis, embryonal leukaemia, eosinophilic leukaemia, Gross' leukaemia, hairy-cell leukaemia, hemoblastic leukaemia, hemocytoblastic leukaemia, histiocytic leukaemia, stem cell leukaemia, acute monocytic leukaemia, leukopenic leukaemia, lymphatic leukaemia, lymphoblastic leukaemia, lymphocytic leukaemia, lymphogenous leukaemia, lymphoid leukaemia, lymphosarcoma cell leukaemia, mast cell leukaemia, megakaryocytic leukaemia, micromyeloblastic leukaemia, monocytic leukaemia, myeloblastic leukaemia, myelocytic leukaemia, myeloid granulocytic leukaemia, myelomonocytic leukaemia, Naegeli leukaemia, plasma cell leukaemia, plasmacytic leukaemia, promyelocytic leukaemia, Rieder cell leukaemia, Schilling's leukaemia, stem cell leukaemia, subleukaemic leukaemia, and undifferentiated cell leukaemia.

The term “lymphoma” generally refers to a malignant neoplasm of the lymphatic system, including cancer of the lymphatic system. The two main types of lymphoma are Hodgkin's disease (HD or HL) and non-Hodgkin's lymphoma (NHL). Abnormal cells appear as congregations which enlarge the lymph nodes, form solid tumours in the body, or more rarely, like leukemia, circulate in the blood. Hodgkin's disease lymphomas, include nodular lymphocyte predominance Hodgkin's lymphoma; classical Hodgkin's lymphoma; nodular sclerosis Hodgkin's lymphoma; lymphocyte-rich classical Hodgkin's lymphoma; mixed cellularity Hodgkin's lymphoma; lymphocyte depletion Hodgkin's lymphoma. Non-Hodgkin's lymphomas include small lymphocytic NHL, follicular NHL; mantle cell NHL; mucosa-associated lymphoid tissue (MALT) NHL; diffuse large cell B-cell NHL; mediastinal large B-cell NHL; precursor T lymphoblastic NEIL; cutaneous T-cell NHL; T-cell and natural killer cell NHL; mature (peripheral) T-cell NHL; Burkitt's lymphoma; mycosis fungoides; Sézary Syndrome; precursor B-lymophoblastic lymphoma; B-cell small lymphocytic lymphoma; lymphoplasmacytic lymphoma; spenic marginal zome B-cell lymphoma; nodal marginal zome lymphoma; plasma cell myeloma/plasmacytoma; intravascular large B-cell NHL; primary effusion lymphoma; blastic natural killer cell lymphoma; enteropathy-type T-cell lymphoma; hepatosplenic gamma-delta T-cell lymphoma; subcutaneous panniculitis-like T-cell lymphoma; angioimmunoblastic T-cell lymphoma; and primary systemic anaplastic large T/null cell lymphoma.

The term “sarcoma” generally refers to a tumour which originates in connective tissue, such as muscle, bone, cartilage or fat, and is made up of a substance like embryonic connective tissue and is generally composed of closely packed cells embedded in a fibrillar or homogeneous substance. Sarcomas include soft tissue sarcomas, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumour sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented haemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumour arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colorectal carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, haematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, non-small cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

As indicated above, the term “carcinoma” also encompasses adenocarcinomas. Adenocarcinomas are carcinomas that originate in cells that make organs which have glandular (secretory) properties or that originate in cells that line hollow viscera, such as the gastrointestinal tract or bronchial epithelia. Examples include, but are not limited to, adenocarcinomas of the breast, lung, pancreas and prostate. In one embodiment of the present invention, the antisense oligonucleotides are used in the treatment of an adenocarcinoma.

Additional cancers encompassed by the present invention include, for example, multiple myeloma, neuroblastoma, rhabdomyosarcoma, primary thrombocytosis, primary macroglobulinemia, small-cell lung tumours, primary brain tumours, malignant pancreatic insulanoma, malignant carcinoid, urinary bladder cancer, premalignant skin lesions, gliomas, testicular cancer, thyroid cancer, esophageal cancer, genitourinary tract cancer, malignant hypercalcemia, endometrial cancer, adrenal cortical cancer, mesothelioma and medulloblastoma.

The cancer to be treated may be indolent or it may be aggressive. The present invention contemplates the use of the antisense oligonucleotides in the treatment of refractory cancers, advanced cancers, recurrent cancers and metastatic cancers. One skilled in the art will appreciate that many of these categories may overlap, for example, aggressive cancers are typically also metastatic.

“Aggressive cancer,” as used herein, refers to a rapidly growing cancer. One skilled in the art will appreciate that for some cancers, such as breast cancer or prostate cancer the term “aggressive cancer” will refer to an advanced cancer that has relapsed within approximately the earlier two-thirds of the spectrum of relapse times for a given cancer, whereas for other types of cancer, such as small cell lung carcinoma (SCLC) nearly all cases present rapidly growing cancers which are considered to be aggressive. The term can thus cover a subsection of a certain cancer type or it may encompass all of other cancer types. A “refractory” cancer or tumour refers to a cancer or tumour that has not responded to treatment. “Advanced cancer,” refers to overt disease in a patient, wherein such overt disease is not amenable to cure by local modalities of treatment, such as surgery or radiotherapy. Advanced disease may refer to a locally advanced cancer or it may refer to metastatic cancer. The term “metastatic cancer” refers to cancer that has spread from one part of the body to another. Advanced cancers may also be unresectable, that is, they have spread to surrounding tissue and cannot be surgically removed.

Certain cancers, such as prostate and breast cancer, can be treated by hormone therapy, i.e. with hormones or anti-hormone drugs that slow or stop the growth of certain cancers by blocking the body's natural hormones. Such cancers may develop resistance, or be intrinsically resistant, to hormone therapy. The present invention further contemplates the use of the antisense oligonucleotides in the treatment of such “hormone-resistant ” or “hormone-refractory” cancers.

Combination Therapy

The present invention also contemplates the use of the one or more of the antisense oligonucleotides in combination therapies with one or more chemotherapeutic agents for the treatment of cancer. The chemotherapeutic agent can be selected from a wide range of cancer chemotherapeutic agents known in the art, including those that target thymidylate synthase. Combinations of standard cancer chemotherapeutics are also known in the art and may be used in conjunction with the antisense oligonucleotides. The present invention further contemplates the use of the antisense oligonucleotides in combination with other antisense oligonucleotides that target thymidylate synthase, but which are complementary to a region of the mRNA other than the coding region. Examples of such antisense oligonucleotides include those described in U.S. Pat. No. 6,087,489, and International Patent Applications WO 99/15648 and WO 98/49287.

As indicated above, during the in vitro and in vivo evaluation of the antisense oligonucleotide, it may be determined that the antisense oligonucleotide is capable of exerting standard antisense effects i.e. decreasing thymidylate synthase mRNA levels, in some cancer cell types. Antisense oligonucleotides against TS that are effective in decreasing levels of TS mRNA have been demonstrated to enhance the effects of TS targeting drugs in cancer cells. Accordingly, one embodiment of the present invention provides for the use of the antisense oligonucleotide in conjunction with one or more chemotherapeutic agent that targets TS. Examples of suitable TS inhibiting chemotherapeutics include, but are not limited to, the fluoropyrimidine drugs 5-FU, 5-FUdR, capecitabine (an oral form of a pro-drug of 5-FU) and a topical 5-FU cream (Effudex®), as well as the non-fluoropyrimidine drugs raltitrexed, methotrexate and pemetrexed (Alimta®). These chemotherapeutic agents are used alone and in combination in a variety of treatment regimens against various tumours including colorectal, breast, lung, cervical and mesothelioma.

One embodiment of the present invention provides for the use of the antisense oligonucleotide in conjunction with one or more chemotherapeutic agent that targets TS in the treatment of colorectal, breast, lung or cervical cancer.

For the treatment of breast cancer, the antisense oligonucleotides can be used in combination with capecitabine (e.g. Xeloda®), cyclophosphamide, 5-fluorouracil (5-FU), carboplatin, paclitaxel (e.g. Taxol®), cisplatin, docetaxel (e.g. Taxotere®), Ifosfamide, Epi-doxorubicin (epirubicin), Doxorubicin (e.g. Adriamycin®), Trastuzumab (Herceptin®) or Tamoxifen, or a combination of these chemotherapeutics, such as the combination of epirubicin with paclitaxel or docetaxel, or the combination of doxorubicin or epirubicin with cyclophosphamide, which are used for breast cancer treatments. Polychemotherapeutic regimens are also useful and may consist, for example, of doxorubicin/cyclophosphamide/5-fluorouracil or cyclophosphamide/epirubicin/5-fluorouracil. Many of the above chemotherapeutics and combinations thereof are useful in the treatment of a variety of solid tumours.

For the treatment of cervical cancer, the antisense oligonucleotides can be used in combination with cisplatin, ifosfamide, fluorouracil or a combination thereof.

5-FU has been used as chemotherapeutic for many years alone and in conjunction with other chemotherapeutics. The following exemplary therapeutic regimens are provided with the understanding that one skilled in the art would appreciate that they may be applied to the situations where 5-FU is used alone or conjunction with another chemotherapeutic. A first exemplary regimen is the Mayo regimen, wherein 1 cycle consists of 5-FU administered at 425 mg/m² by intravenous bolus injection daily together with 20 mg/m² leucovorin for 5 days, followed by 3 weeks off. A second therapeutic regimen may consist of administering 200 to 220 mg/m² 5-FU by continuous infusion over 24 hours once a week. A third therapeutic regimen consists of shorter, intermittent infusions of 5-FU from between 24 to 120 hours, every week, two weeks, three weeks or four weeks at dosages of 600 mg/m² to 2500 mg/m² per 24 hours. One skilled in the art will also appreciate that 5-FU and its variants can be used in combination therapies with a variety of other traditional chemotherapeutic drugs.

An exemplary therapeutic regimen for raltitrexed (Tomudex®) is administration at 3 mg/m² once every 3 weeks by bolus injection.

An exemplary regimen for pemetrexed is administration at 500 mg/m2 once every 3 weeks. Pemetrexed may be used in this regimen alone or in combination with cisplatin. Examples of additional supportive drugs that could be included in the above regimen include: folic acid daily at 0.4 mg, Vitamin B12 at 1000 micrograms every 9 weeks. Dexamethazone may also be included as a supportive drug.

Other chemotherapeutic agents contemplated by the present invention include those which may be applicable to a range of cancers, such as doxorubicin, capecitabine, mitoxantrone, irinotecan (CPT-11), as well as those that are suited to the treatment of a specific cancer.

Cyclophosphamide, mitoxantrone and estramustine are known to be suitable for the treatment of prostate cancer. Cyclophosphamide, vincristine, doxorubicin and etoposide are used in the treatment of small cell lung cancer, as are combinations of etoposide with either cisplatin or carboplatin. In the treatment of stomach or oesophageal cancer, combinations of doxorubicin or epirubicin with cisplatin and 5-fluorouracil are useful. For colorectal cancer, CPT-11 alone or in combination with 5-fluorouracil-based drugs, or oxaliplatin alone or in combination with 5-fluorouracil-based drugs can be used. Oxaliplatin may also be used in combination with capecitabine.

Other examples include the combination of cyclophosphamide, doxorubicin, vincristine and prednisone in the treatment of non-Hodgkin's lymphoma; the combination of doxorubicin, bleomycin, vinblastine and DTIC in the treatment of Hodgkin's disease and the combination of cisplatin or carboplatin with any one or a combination of gemcitabine, paclitaxel, docetaxel, vinorelbine or etoposide in the treatment of non-small cell lung cancer. Pemetrexed alone is also a proven effective drug in the treatment of non-small cell lung cancer. Other suitable chemotherapeutic agents include, but are not limited to, mitomycin C, vinblastine, IL-2, novantrone, DTIC and hydroxyurea.

One embodiment of the present invention contemplates the use of the antisense oligonucleotides as “sensitizing agents,” or “chemopetentiators,” which selectively inhibit the growth of cancer cells. In this case, the antisense oligonucleotides alone does not have a cytotoxic effect on the cancer cell, but provides a means of weakening the cancer cells, and thereby facilitates the benefit from conventional anti-cancer therapeutics.

Administration

Typically in the treatment of cancer, therapeutic compounds are administered systemically to patients, for example, by bolus injection or continuous infusion into a patient's bloodstream.

The antisense oligonucleotides may be used as part of a neo-adjuvant therapy (to primary therapy), as part of an adjuvant therapy regimen, where the intention is to cure the cancer in a subject. The present invention contemplates the use of the antisense oligonucleotides at various stages in tumour development and progression, including in the treatment of advanced and/or aggressive neoplasias (i.e. overt disease in a subject that is not amenable to cure by local modalities of treatment, such as surgery or radiotherapy), metastatic disease, locally advanced disease and/or refractory tumours (i.e. a cancer or tumour that has not responded to treatment).

“Primary therapy” refers to a first line of treatment upon the initial diagnosis of cancer in a subject. Exemplary primary therapies may involve surgery, a wide range of chemotherapies and radiotherapy. “Adjuvant therapy” refers to a therapy that follows a primary therapy and that is administered to subjects at risk of relapsing. Adjuvant systemic therapy is begun soon after primary therapy to delay recurrence, prolong survival or cure a subject.

The dosage to be administered is not subject to defined limits, but will be an effective amount to achieve the desired pharmacological and physiological effects. The compositions may be formulated in a unit dosage form. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. Examples of ranges for the compound(s) in each dosage unit are from about 0.05 to about 100 mg, or more usually, from about 1.0 to about 30 mg.

Daily dosages of the compounds of the present invention will typically fall within the range of about 0.01 to about 100 mg/kg of body weight, in single or divided dose. However, it will be understood that the actual amount of the compound(s) to be administered will be determined by a physician, in the light of the relevant circumstances, including the condition to be treated, the chosen route of administration, the actual compound administered, the age, weight, and response of the individual patient, and the severity of the patient's symptoms. The above dosage range is given by way of example only and is not intended to limit the scope of the invention in any way. In some instances dosage levels below the lower limit of the aforesaid range may be more than adequate, while in other cases still larger doses may be employed without causing harmful side effects, for example, by first dividing the larger dose into several smaller doses for administration throughout the day.

When used in conjunction with one or more known chemotherapeutic agents, the antisense oligonucleotides can be administered prior to, or after, administration of the chemotherapeutic agents, or they can be administered concomitantly. The one or more chemotherapeutic may be administered systemically, for example, by bolus injection or continuous infusion, or it may be administered orally.

Clinical Trials in Cancer Patients

One skilled in the art will appreciate that, for the treatment of human patients, the antisense oligonucleotides, alone or in combination with one or more chemotherapeutic agents, should be tested in Clinical Trials in order to further evaluate their efficacy in the treatment of cancer and to obtain regulatory approval for therapeutic use. As is known in the art, clinical trials progress through phases of testing, which are identified as Phases I, II, III, and IV.

Initially the antisense oligonucleotides will be evaluated in a Phase I trial. Typically

Phase I trials are used to determine the best mode of administration (for example, by pill or by injection), the frequency of administration, and the toxicity for the compounds. Phase I studies frequently include laboratory tests, such as blood tests and biopsies, to evaluate the effects of a compound in the body of the patient. For a Phase I trial, a small group of cancer patients are treated with a specific dose of the antisense oligonucleotide(s). During the trial, the dose is typically increased group by group in order to determine the maximum tolerated dose (MTD) and the dose-limiting toxicities (DLT) associated with the compound. This process determines an appropriate dose to use in a subsequent Phase II trial.

A Phase II trial can be conducted to evaluate further the effectiveness and safety of the antisense oligonucleotides. In Phase II trials, the antisense oligonucleotide is administered to groups of patients with either one specific type of cancer or with related cancers, using the dosage found to be effective in Phase I trials.

Phase III trials focus on determining how a compound compares to the standard, or most widely accepted, treatment. In Phase III trials, patients are randomly assigned to one of two or more “arms”. In a trial with two arms, for example, one arm will receive the standard treatment (control group) and the other arm will receive treatment with the antisense oligonucleotide (investigational group).

Phase IV trials are used to further evaluate the long-term safety and effectiveness of a compound. Phase IV trials are less common than Phase I, II and III trials and will take place after the antisense oligonucleotide has been approved for standard use.

Eligibility of Patients for Clinical Trials

Participant eligibility criteria can range from general (for example, age, sex, type of cancer) to specific (for example, type and number of prior treatments, tumour characteristics, blood cell counts, organ function). Eligibility criteria may also vary with trial phase. For example, in Phase I and II trials, the criteria often exclude patients who may be at risk from the investigational treatment because of abnormal organ function or other factors. In Phase II and III trials additional criteria are often included regarding disease type and stage, and number and type of prior treatments.

Phase I cancer trials usually comprise 15 to 30 participants for whom other treatment options have not been effective. Phase II trials typically comprise up to 100 participants who have already received chemotherapy, surgery, or radiation treatment, but for whom the treatment has not been effective. Participation in Phase II trials is often restricted based on the previous treatment received. Phase III trials usually comprise hundreds to thousands of participants. This large number of participants is necessary in order to determine whether there are true differences between the effectiveness of the antisense oligonucleotides and the standard treatment. Phase III may comprise patients ranging from those newly diagnosed with cancer to those with extensive disease in order to cover the disease continuum.

One skilled in the art will appreciate that clinical trials should be designed to be as inclusive as possible without making the study population too diverse to determine whether the treatment might be as effective on a more narrowly defined population. The more diverse the population included in the trial, the more applicable the results could be to the general population, particularly in Phase III trials. Selection of appropriate participants in each phase of clinical trial is considered to be within the ordinary skills of a worker in the art.

Assessment of Patients Prior to Treatment

Prior to commencement of the study, several measures known in the art can be used to first classify the patients. Patients can first be assessed, for example, using the

Eastern Cooperative Oncology Group (ECOG) Performance Status (PS) scale or the Karnofsky Performance Status (KPS) scale, both of which are widely accepted standards for the assessment of the progression of a patient's disease as measured by functional impairment in the patient.

Patients' overall quality of life can be assessed, for example, using the McGill Quality of Life Questionnaire (MQOL) (Cohen et al (1995) Palliative Medicine 9: 207-219). The MQOL measures physical symptoms; physical, psychological and existential well-being; support; and overall quality of life. To assess symptoms such as nausea, mood, appetite, insomnia, mobility and fatigue the Symptom Distress Scale (SDS) developed by McCorkle and Young ((1978) Cancer Nursing 1: 373-378) can be used.

Patients can also be classified according to the type and/or stage of their disease and/or by tumour size.

Administration of the Antisense Oligonucleotide in Clinical Trials

The antisense oligonucleotide is typically administered to the trial participants parenterally. In one embodiment, the antisense oligonucleotide is administered by intravenous infusion. Methods of administering drugs by intravenous infusion are known in the art. Usually intravenous infusion takes place over a certain time period, for example, over the course of 60 minutes. In other embodiments of the invention, for example, for the treatment of patients with mesothelioma, the antisense oligonucleotide is administered intracavitorially, i.e. by intrapleural, intraperitoneal or intrapericardial infusion.

Monitoring of Patient Outcome

The endpoint of a clinical trial is a measurable outcome that indicates the effectiveness of a treatment under evaluation. The endpoint is established prior to the commencement of the trial and will vary depending on the type and phase of the clinical trial. Examples of endpoints include, for example, tumour response rate—the proportion of trial participants whose tumour was reduced in size by a specific amount, usually described as a percentage; disease-free survival—the amount of time a participant survives without cancer occurring or recurring, usually measured in months; overall survival—the amount of time a participant lives, typically measured from the beginning of the clinical trial until the time of death. For advanced and/or metastatic cancers, disease stabilisation—the proportion of trial participants whose disease has stabilised, for example, whose tumour(s) has ceased to grow and/or metastasise, can be used as an endpoint. Other endpoints include toxicity and quality of life.

Tumour response rate is a typical endpoint in Phase II trials. However, even if a treatment reduces the size of a participant's tumour and lengthens the period of disease-free survival, it may not lengthen overall survival. In such a case, side effects and failure to extend overall survival might outweigh the benefit of longer disease-free survival. Alternatively, the participant's improved quality of life during the tumour-free interval might outweigh other factors. Thus, because tumour response rates are often temporary and may not translate into long-term survival benefits for the participant, response rate is a reasonable measure of a treatment's effectiveness in a Phase II trial, whereas participant survival and quality of life are typically used as endpoints in a Phase III trial.

Pharmaceutical Kits

The present invention additionally provides for therapeutic kits containing the antisense oligonucleotide and optionally one or more chemotherapeutic agents for use in the treatment of cancer. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. In this case the container means may itself be an inhalant, syringe, pipette, eye dropper, or other such like apparatus, from which the composition may be administered to a subject.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a subject. Such an instrument may be an inhalant, syringe, pipette, forceps, measured spoon, eye dropper or any such medically approved delivery vehicle.

To gain a better understanding of the invention described herein, the following examples are set forth. It should be understood that these examples are for illustrative purposes only. Therefore, they should not limit the scope of this invention in any way.

### Examples

For examples 1 to 6 below, the following materials and methods were employed.

Cell Culture

HeLa and MCF-7 cells were obtained from the American Type Culture Collection (Manassas, Va.). HeLa cells were grown in Dulbecco's modified Eagle's medium and MCF-7 cells were grown in a-minimal essential media plus, both containing 10% fetal bovine serum (FBS), at 37° C. in a humified atmosphere of 5% CO₂. Tissue culture reagents were from Invitrogen Canada (Burlington, ON, Canada).

Oligodeoxynucleotides

The following antisense oligonucleotides were compared:

| | |
|---|---|
| 5′-CAGCGGAGGATGTGTTGGAT-3′ | [SEQ ID NO: 2] |

is complementary to bases 214 to 233 within the human TS coding region.

| | |
|---|---|
| 5′-GGAGTGCGTGAGTCGATGTA-3′ | [SEQ ID NO: 6] |

is a control scrambled oligonucleotide that has the same base composition as SEQ ID NO:2 in a random order.

| | |
|---|---|
| 5′-CGGCACGCCCATAGGCGGCG-3′ | [SEQ ID NO: 3] |

also used as a control, has a similar randomized base composition.

| | |
|---|---|
| 5′-GCCAGTGGCAACATCCTTAA-3′ | [SEQ ID NO: 4] |

is complementary to bases 1184 to 1203 within the human TS 3′-UTR.

| | |
|---|---|
| 5′-ATGGCGCCAACGGTTCCTAAA-3′ | [SEQ ID NO: 5] |

is a control scrambled oligonucleotide that has the same base composition as SEQ ID NO:4 in a random order.

| | |
|---|---|
| 5′-GGCCGGCGCGGCAGCTCCGA-3′ | [SEQ ID NO: 7] |

is complementary to bases 121 to 140 within the human TS coding region.

| | |
|---|---|
| 5′-GCAGCTCCGAGCCCGGCCACA-3′ | [SEQ ID NO: 8] |

is complementary to bases 111 to 130 within the human TS coding region.

| | |
|---|---|
| 5′-CATGCCGAATACCGACAGGG-3′ | [SEQ ID NO: 9] |

is complementary to bases 269 to 278 within the TS coding region.

All of the oligonucleotides have phosphorothioated internucleotide linkages. The 6 nucleotides at both the 5′ and 3′ ends of SEQ ID NOs:2, 3, 4, 5, 7, 8 and 9 can be modified on the 2′ position of the ribose with 2′-methoxyethoxy (oligonucleotides were provided by ISIS Pharmaceuticals, Carlsbad, Calif.). The 6 nucleotides at both the 5′ and 3′ ends of SEQ ID NOs:2, 4, 5 and 6 can be modified on the 2′ position of the ribose with 2′-O-methyl (oligonucleotides purchased from Eurogentec North America, San Diego, Calif.). Essentially identical results were obtained in experiments using oligoncleotides with either 2′ ribose modification. There was no effect of SEQ ID NO:3, 5 or 6 on proliferation, cell death, TS mRNA or protein levels in either cell line.

Cell Proliferation Assay

Cells were plated at a density of 1×10⁵ cells/25-cm² flask. The following day, oligonucleotides (50 nM) were mixed with Lipofectamine 2000 (0.5 μg/ml) in serum-free medium for 20 minutes, after which FBS (0.1 volumes) was added. The medium on the cells was then replaced with 2 ml of medium with ODN/lipid. Four hours later, a further 2 ml of growth medium was added, and the cells were incubated for up to 4 days. Cells were removed from the flasks by trypsin treatment, and counted in saline solution using an electronic particle counter (Beckman Coulter, Hialeah, Fla.) at the time of oligonucleotide treatment (day 0), and each day for 4 days thereafter. Proliferation rate was calculated using the formula: (final-initial)/initial, and, where indicated, is expressed as a percentage of proliferation in the presence of the control oligonucleotide. For drug sensitivity assays, cells were treated with oligonucleotides as above. After the initial 4 hour oligonucleotide treatment, the appropriate concentration of drug was added in the 2 ml aliquot of growth medium. The cells were incubated for 4 days and counted and proliferation is expressed relative to treatment with oligonucleotide alone.

RNA and Protein Isolation and Analysis

Cells were treated with oligonucleotides as described above, except that cells were plated at a density of 2×10⁶/75-cm² flask, and treated with 100 nM oligonucleotide and 1 μg/ml Lipofectamine 2000. After 24 or 48 hours, cells were washed with cold PBS and collected by scraping with a rubber policeman. Cells were centrifuged at 200×g for 10 minutes. The cell pellets were washed a second time in cold PBS and centrifuged again. Total RNA was isolated using TRIzol (Invitrogen, Canada) and quantitated using a spectrophotometer. Reverse transcription of 1 μg of RNA using MMLV-RT (Invitrogen, Canada) was followed by PCR using primers specific for GAPDH and TS as described (Berg R W, et al., J Pharm Exp Ther 2001; 298: 477-484; Berg R W, et al., Cancer Gene Ther 2003; 10:278-286). PCR products were visualized on 1% agarose gels stained with ethidium bromide. The DNA was then transferred by Southern blotting to Hybond-N (Amersham Pharmacia Biotech AB, Uppsala, Sweden) and hybridized with ³²P-labeled GAPDH or TS cDNA for more accurate quantitation. To quantitate TS protein activity, a [³H]-FdUMP binding assay was used, exactly as described (Ferguson P J, et al., Br J Pharmacol 1999; 127:1777-1786).

ODN Uptake Measurement

Cells were plated at 1×10⁴ cells/well in 24-well plates and incubated for 24 hours. SEQ ID NO:3 was end-labeled with [γ³²P]-ATP using 20 units of T4 polynucleotide kinase (Invitrogen, Canada) for 10 minutes at 37° C., and unincorporated radionucleotide removed using a Sephadex G50 NICK column (Amersham Pharmacia Biotech AB). Labeled SEQ ID NO:3 (˜1×10⁶ cpm) was mixed with unlabeled SEQ ID NO:3 (50 nM) and Lipofectamine 2000 (0.5 μg/ml) as above, and added to each well. After 24 hours, the medium was removed and cells washed 3 times with PBS. The cells were lysed (10 mM Tris-Cl, pH 8.0; 0.1 M EDTA, pH 5.0; 10% w/v SDS) and radioactivity measured by scintillation counting and by Phosphorimager analysis (Molecular Dynamics, Sunnyvale, Calif.) following dot-blotting to Hybond N.

Flow Cytometry

Cells were plated at a density of 1×10⁵ cells/25-cm² flask. The following day, cells were treated with oligonucleotides (50 nM) as described above and incubated for up to 4 days. At each time-point, media was collected, cells were washed with PBS and trypsinized. Cells (both adherent and nonadherent) were then combined in 2.5 ml of media and centrifuged at 200×g for 10 minutes at 4° C. Supernatant was removed and cells were washed with 4 mL of cold PBS and re-centrifuged. Binding buffer (0.14 M NaCl; 2.5 mM CaCl₂; 10 mM HEPES, pH 7.4) and staining solution (Annexin V-FITC, BD Sciences and propidium iodide, 50 μg/mL in PBS) was mixed with 1×10⁵ cells and incubated for 15 minutes. Flow cytometry was carried out on a Beckman Coulter Epics XL-MCL Flow Cytometer with at least 10,000 events collected. For cell cycle analysis cells were plated at 2.5×10⁵ cells/75-cm² flask, oligonucleotide treatment was carried out as described above, and cell cycle analysis carried out by the procedure supplied by Becton-Dickinson.

Statistical Analysis

Statistical significance within experiments was determined using Student's t-test (P<0.05). All experiments were performed at least three times.

### Example 1

### Differential Effect of SEQ ID NO:2 on Proliferation, TS mRNA and TS Protein in HeLa and MCF-7 Cells

SEQ ID NO:4, which targets the 3’-UTR of TS mRNA, has been previously demonstrated to inhibit proliferation of HeLa and HT-29 cells (Ferguson P J, et al., Br J Pharmacol 1999; 127:1777-1786; Ferguson P J, et al., Br J Pharmacol 2001; 134:1437-1446; Berg R W, et al., J Pharm Exp Ther 2001; 298:477-484; Berg R W, et al., Cancer Gene Ther 2003; 10:278-286). To determine whether sequences within the coding region of TS mRNA were similarly sensitive to AS targeting to inhibit cell proliferation, HeLa and MCF-7 cells were treated with a panel of TS AS oligonucleotides (including SEQ ID NO:2), chosen on the basis of unique sequence and GC content, and cell numbers were assessed at various times after treatment (see Table 2).

TABLE 2
MCF-7 Cell Proliferation after Treatment with Oligonucleotides

Cells per Flask¹
(average ± SD)

| Experiment | SEQ ID NO | Day 2 | Day 3 |
|---|---|---|---|
| I | 3 | 15.3 ± 2.77 | 32.8 ± 0.98 |
| | 7 | 15.7 ± 1.50 | 22.4 ± 1.96 |
| | 8 | 19.2 ± 0.16 | 26.5 ± 1.56 |
| | 2 | 2.8 ± 0.26 | 2.2 ± 0.26 |
| | 9 | 13.7 ± 0.94 | 24.3 ± 1.07 |
| II | 5 | 27.4 ± 5.44 | 51.8 ± 4.50 |
| | 4 | 12.7 ± 1.98 | 9.6 ± 1.46 |

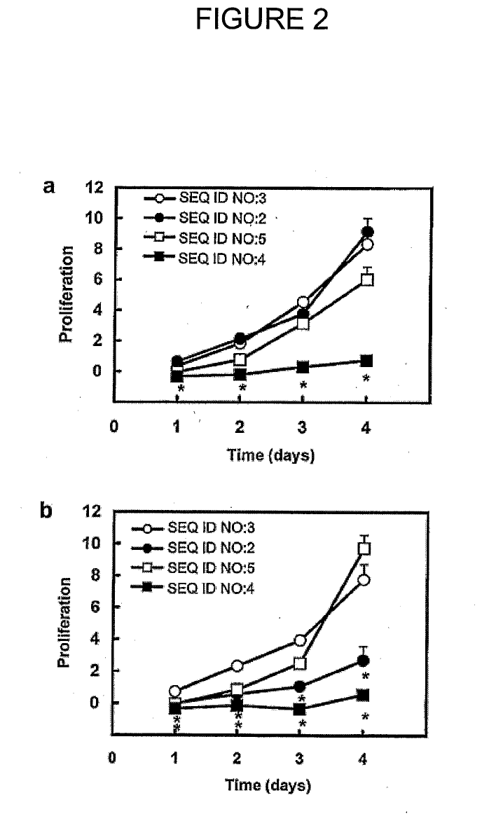

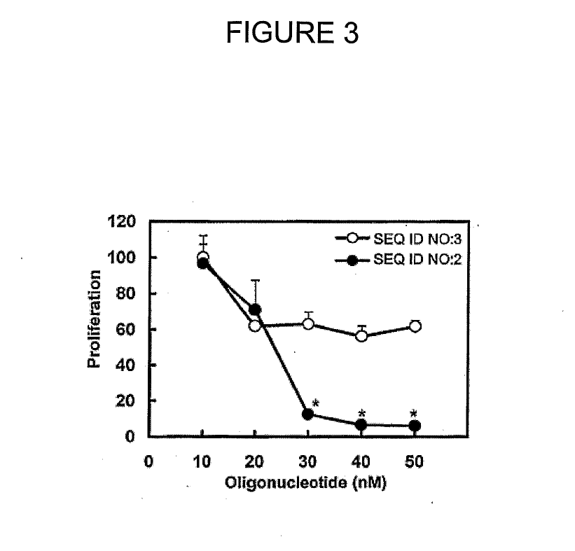

The effect of SEQ ID NO:2 in the inhibition of proliferation of HeLa and MCF-7 cells was further evaluated. The results are shown in FIGS. 2 and 3. FIG. 2 shows results from the treatment of (A) HeLa cells (1×10⁵) and (B) MCF-7 cells (1×10⁵) with 50 nM control SEQ ID NO:3 (∘), SEQ ID NO:5 (□), SEQ ID NO:2 () or SEQ ID NO:4 (▪) as described in Materials and Methods. Cells were counted, in triplicate, on days 1 through 4 after ODN addition. Fold increases in cell number (relative to starting cell number) are shown. FIG. 3 shows results from the treatment of MCF-7 cells (1×10⁵) with the indicated concentrations of control SEQ ID NO:3 (∘) or SEQ ID NO:2 () as described. Cells were counted, in triplicate, on day 4 after oligonucleotide addition. Data points indicate mean values +SD. Asterisks (*) indicate values significantly different from cells treated with control oligonucleotides (n=3, P<0.05, Student's t-test).

Treatment with SEQ ID NO:2 caused no change in proliferation of HeLa cells, compared to cells treated with the control SEQ ID NO:3 (FIG. 2A). In contrast, SEQ ID NO:2 (but not control SEQ ID NO:3) reduced proliferation of MCF-7 cells in a dose-dependent fashion (FIG. 3). Treatment with SEQ ID NO:2 (30 to 50 nM) of MCF-7 cells inhibited cell proliferation by 65 to 90% (FIG. 2B and FIG. 3), whereas 10 or 20 nM SEQ ID NO:2 did not significantly reduce proliferation relative to the scrambled control (FIG. 3). Inhibition of HeLa cell proliferation by SEQ ID NO:4 was included as a positive control (FIG. 2A), and SEQ ID NO:4 likewise inhibited MCF-7 proliferation, compared to its control SEQ ID NO:5 (FIG. 2B).

### Example 2

### Differential Effect of SEQ ID NO:2 on TS mRNA and TS Protein in HeLa and MCF-7 Cells

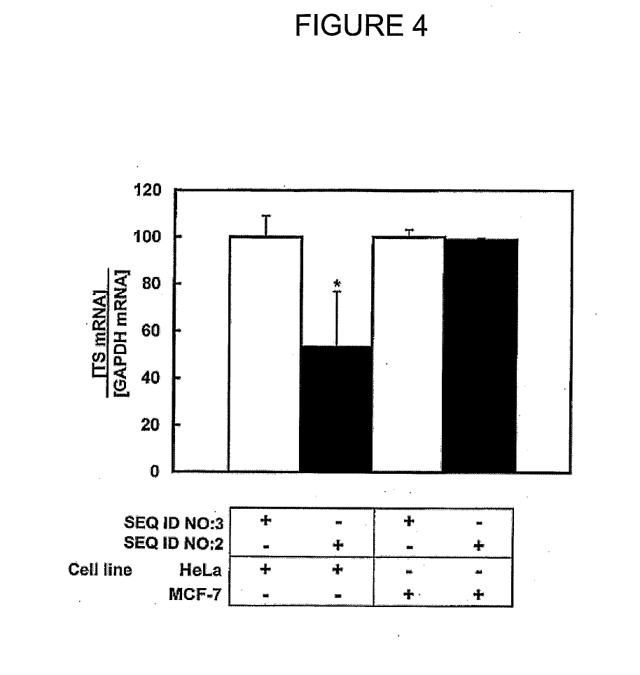

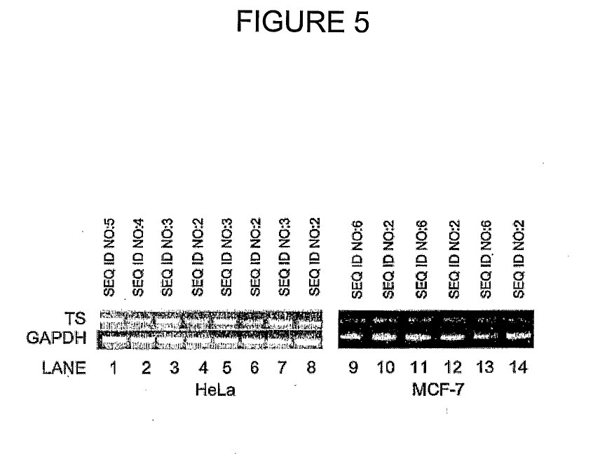

The effect of SEQ ID NO:2 on TS mRNA and protein levels was also investigated in HeLA and MCF-7 cells. The results are shown in FIGS. 4 and 5. FIG. 4A shows the results of (A) treatment of HeLa and MCF-7 cells with control SEQ ID NO:3 (100 nM, open bars) or SEQ ID NO:2 (100 nM, black bars) for 24 hours as described in Materials and Methods. TS mRNA/GAPDH mRNA values were normalized to 1.0 for cells treated with SEQ ID NO:3, and values derived from cells treated with SEQ ID NO:2 are presented relative to those normalized values. FIG. 4B shows agarose gels with RT-PCR products of TS and GAPDH mRNA from representative experiments where HeLa cells were treated with control SEQ ID NO:5 or SEQ ID NO:4 (lanes 1,2) or with control SEQ ID NO:3 or SEQ ID NO:2 (triplicate experiment, lanes 3-8), and where MCF-7 cells were treated with control SEQ ID NO:6 or SEQ ID NO:2 (triplicate experiment, lanes 9-14). Asterisk (*) indicates significantly different value from cells treated with control SEQ ID NO:3 (n=3, P=0.048, Student's t-test).

Reduced proliferation of HeLa cells treated with SEQ ID NO:4 is associated with reduced TS mRNA (Ferguson P J, et al., Br J Pharmacol 1999; 127:1777-1786; Ferguson P J, et al., Br J Pharmacol 2001; 134:1437-1446). Similarly, treatment of HeLa cells with SEQ ID NO:2 for 24 hours reduced the relative TS mRNA level ([TS mRNA]/[GAPDH mRNA]) by 50% compared to cells treated with control SEQ ID NO:3 (FIG. 4). In contrast, there was no difference in TS mRNA level (relative to GAPDH mRNA) between MCF-7 cells treated with SEQ ID NO:2 or with control SEQ ID NO:6 (FIG. 5). Reduction of TS mRNA in HeLa cells by treatment with SEQ ID NO:2 is transient: 48 hours after treatment, relative TS mRNA levels in SEQ ID NO:2 and control SEQ ID NO:3 treated HeLa cells were not different ([TS mRNA]/[GAPDH mRNA]=0.728±0.03 for SEQ ID NO:2, and 0.846±0.07 for SEQ ID NO:3, P=0.51). There is a similar transient reduction in TS mRNA in HeLa cells after SEQ ID NO:4 treatment (Ferguson P J, et al., Br J Pharmacol 1999; 127:1777-1786; Ferguson P J, et al., Br J Pharmacol 2001; 134:1437-1446; Berg R W, et al., J Pharm Exp Ther 2001; 298:477-484).

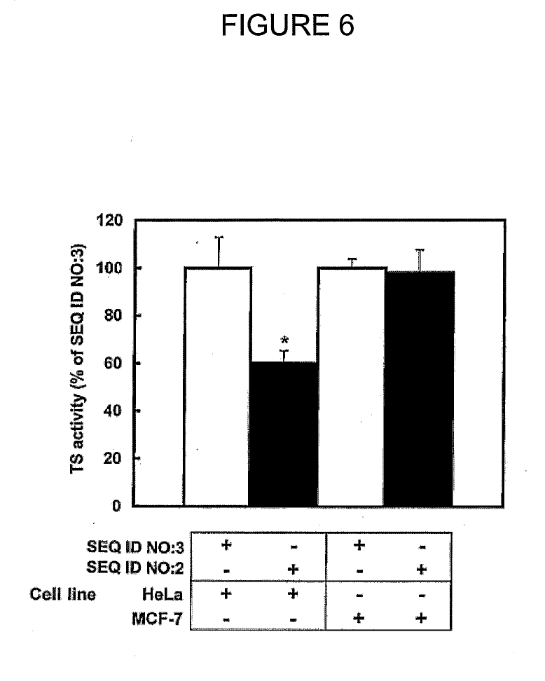

In HeLa cells treated with SEQ ID NO:4, reduced TS mRNA levels correlated with reduced TS protein level and activity (Ferguson P J, et al., Br J Pharmacol 1999; 127:1777-1786; Ferguson P J, et al., Br J Pharmacol 2001; 134:1437-1446). To determine whether SEQ ID NO:2 treatment of HeLa and MCF-7 cells similarly reduced TS protein levels, a [6-³H]-FdUMP binding assay was used to measure TS protein activity (i.e., capacity to bind uridine monophosphate). FIG. 6 shows the results of treatment of HeLa and MCF-7 cells with control SEQ ID NO:3 (100 nM, open bars) or SEQ ID NO:2 (100 nM, black bars) for 24 hours as described in Materials and Methods. TS protein activity in cell lysates was measured by [6-³H]-FdUMP binding. Asterisk (*) indicates significantly different value from cells treated with control SEQ ID NO:3 (n=3, P=0.008, Student's t-test).

In extracts of HeLa cells treated with SEQ ID NO:2 for 24 hours, [6-³H]-FdUMP binding was decreased by 40% compared to cells treated with control SEQ ID NO:3 (FIG. 6). There was no difference in [6-³H]-FdUMP binding between extracts of MCF-7 cells treated with SEQ ID NO:2 or control SEQ ID NO:3 (FIG. 5). Thus, TS protein activity paralleled TS mRNA levels after SEQ ID NO:2 treatment in both HeLa and MCF-7 cells.

### Example 3

### Uptake of Oligonucleotides in HeLa and MCF-7 Cell Lines

To investigate whether the apparent cell-specific behaviour of SEQ ID NO:2 could be attributed to differential uptake of oligonucleotides between cell lines, HeLa and MCF-7 cells were treated with [³²P]-end-labeled SEQ ID NO:3, and radioactivity associated with the cells (i.e., oligonucleotide uptake) was measured. There was no difference in the amount of labeled oligonucleotide associated with HeLa (293±87) or MCF-7 (234±71) cells (arbitrary units, n=3, P=0.23). In situ hybridization experiments detected SEQ ID NO:4 and scrambled control SEQ ID NO:5 in 96±1.4% of HeLa cells (n=4 experiments, >150 cells counted in each) and 74±7.9% of MCF-7 cells (n=8 experiments, >150 cells counted in each). Therefore, it appears that these two cell lines are nearly equivalent in their ability to internalize oligonucleotides under the transfection conditions used in these studies.

### Example 4

### SEQ ID NO:2 Increases HeLa Cell Sensitivity to TS-Targeting Chemotherapeutic Drugs

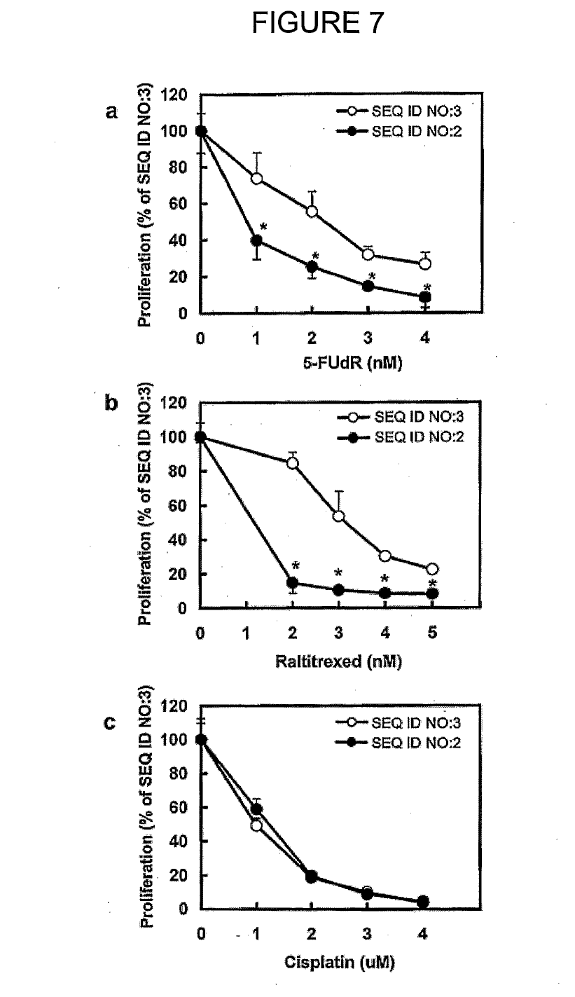

It has been previously demonstrated that SEQ ID NO:4 sensitizes HeLa cells to TS-targeting chemotherapy drugs (including 5-FUdR and raltitrexed), but not to non-TS-targeting drugs such as cisplatin (Ferguson P J, et al., Br J Pharmacol 1999; 127:1777-1786; Ferguson P J, et al., Br J Pharmacol 2001; 134:1437-1446). To determine whether SEQ ID NO:2 altered human tumor cell sensitivity to chemotherapeutic drugs, HeLa and MCF-7 cells were treated with SEQ ID NO:2 in combination with 5-FUdR, raltitrexed or cisplatin. FIG. 7 shows the results of treatment of HeLa cells with 50 nM control SEQ ID NO:3 (∘) or SEQ ID NO:2 () as described in Materials and Methods. The indicated concentrations of 5-FUdR, raltitrexed or cisplatin were added, and the cells were incubated for 4 days. Proliferation relative to cells treated with SEQ ID NO:2 or SEQ ID NO:3 in the absence of drug is shown. Asterisks (*) denote significant differences in proliferation after treatment with SEQ ID NO:2 compared with control SEQ ID NO:3 at the same dose of 5-FUdR or raltitrexed (n=3, P<0.05, Student's t-test).

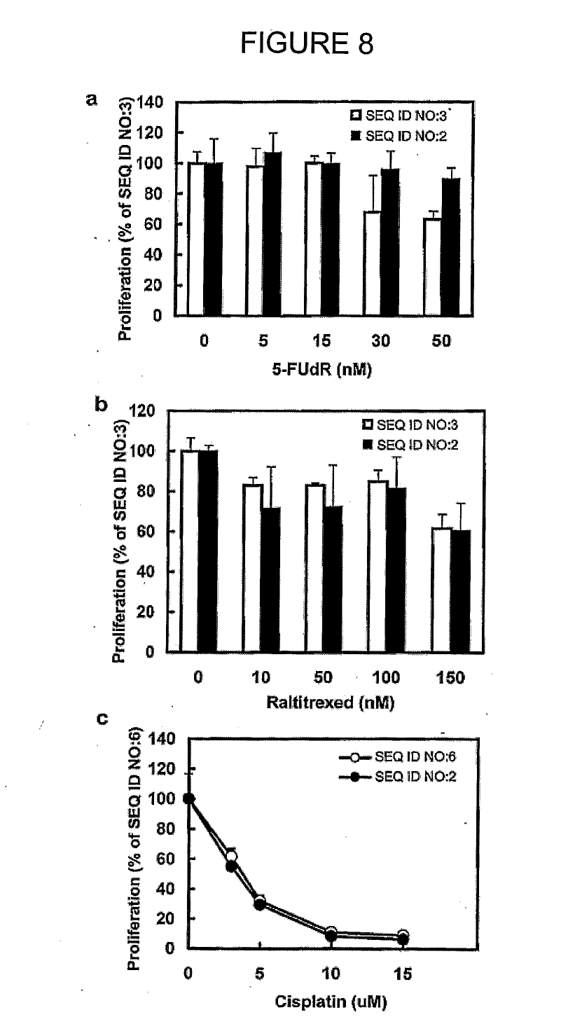

FIG. 8 shows the results of treatment of MCF-7 cells with 10 nM control SEQ ID NO:3 (□) or SEQ ID NO:6 (∘) or SEQ ID NO:2 (▪,) as described in Materials and Methods. The indicated concentrations of 5-FUdR, raltitrexed or cisplatin were added, and the cells were incubated for 4 days. Proliferation rate (relative to cells treated with SEQ ID NO:2 or SEQ ID NO:6 in the absence of drug) is shown (n=3, P>0.05, Student's t-test).

SEQ ID NO:2 (10 or 50 nM) increased the cytotoxic effect of 5-FUdR (FIG. 7A), raltitrexed (FIG. 7B) and 5-FU in HeLa cells, whereas cisplatin sensitivity was not affected (FIG. 7C). In contrast, SEQ ID NO:2 (10 nM) did not alter MCF-7 cell sensitivity to 5-FUdR (FIG. 8A) or raltitrexed (FIG. 8B). Since SEQ ID NO:2 (50 nM) on its own completely inhibited MCF-7 cell proliferation (FIG. 2B and FIG. 3), the capacity to enhance drug sensitivity at the higher dose could not be assessed in MCF-7 cells. As with HeLa cells, cisplatin cytotoxicity was equivalent in MCF-7 cells after treatment with SEQ ID NO:2 or control SEQ ID NO:6 (FIG. 8C). Thus, reduction of TS mRNA and TS protein, and chemosensitization by SEQ ID NO:2 treatment occurred in HeLa but not MCF-7 cells, and chemosensitization was restricted to those chemotherapy drugs that target TS.

### Example 5

### SEQ ID NO:2 Induces Apoptosis in MCF-7 but not HeLa Cells

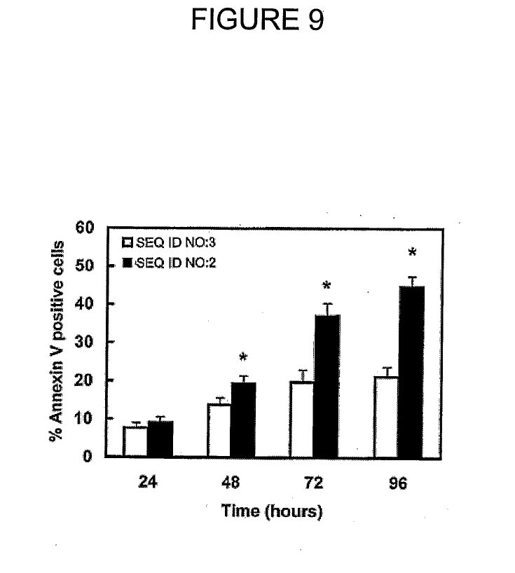

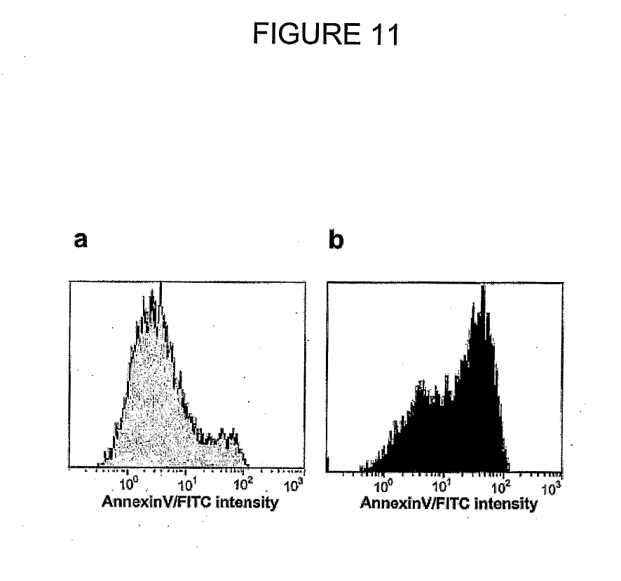

SEQ ID NO:4 has been previously shown to induce G₂/M cell cycle arrest without apoptosis in HeLa and HT-29 cells (Berg R W, et al., J Pharm Exp Ther 2001; 298: 477-484). To determine if the robust antiproliferative effect of SEQ ID NO:2 in MCF-7 cells was due to increased apoptosis and/or necrotic cell death, oligonucleotide-treated cells were subjected to flow cytometric analysis after staining with Annexin V and propidium iodide. FIGS. 9 and 11 show the results from treatment of MCF-7 cells with 50 nM control SEQ ID NO:3 (open bars in FIG. 9) or SEQ ID NO:2 (filled bars in FIG. 9) and incubated for 1 to 4 days. Cells were collected and stained as described in Materials and Methods. For each condition, 10 000 events were collected and analyzed. Percent of cells actively undergoing apoptosis (i.e., annexin V positive, propidium iodide negative) is shown. Asterisks (*) denote significant differences compared with control SEQ ID NO:3 (n=3, P<0.05, Student's t-test).

At 48, 72 and 96 hours after treatment of MCF-7 cells with SEQ ID NO:2 there was a significant increase in Annexin V staining (i.e. cells in early apoptosis) compared to cells treated with control SEQ ID NO:3 (FIG. 9). In contrast, SEQ ID NO:2 did not induce HeLa cell apoptosis (16:2%±1.5% vs 15.1%±0.8% Annexin V positivity in HeLa cells treated for 72 hours with SEQ ID NO:2 vs scrambled control SEQ ID NO:3, n=3, P=0.319), consistent with the previous report that SEQ ID NO:4 treatment did not induce apoptosis (Berg R W, et al., Cancer Gene Ther 2003; 10:278-286).

### Example 6

### Cell Cycle Effect in MCF-7 and HeLa Cells

As noted above, SEQ ID NO:4 has been previously shown to induce G₂/M cell cycle arrest without apoptosis in HeLa and HT-29 cells (Berg R W, et al., J Pharm Exp Ther 2001; 298: 477-484). Accordingly, the effect of SEQ ID NO:2 on cell cycle in MCF-7 and HeLa cells was investigated as described in the Materials and Methods.

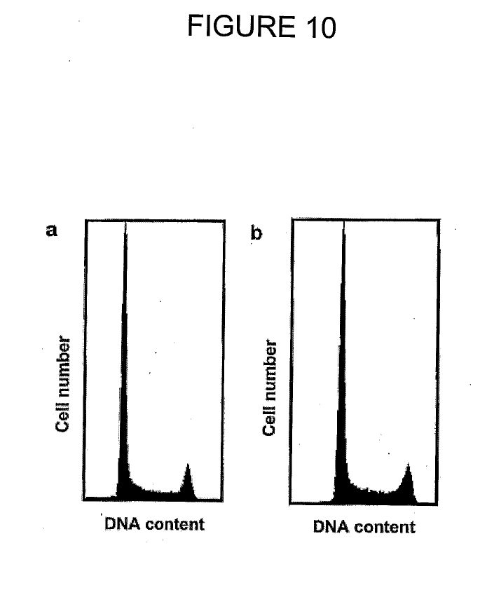

FIG. 10 shows the results from treatment of HeLa cells with 100 nM control SEQ ID NO:3 (Panel A) or SEQ ID NO:2 (Panel B) and incubated for 48 hours. Cells were collected and stained with propidium iodide as described in Materials and Methods. For each condition, 50 000 events were collected and analysed. The histograms show numbers of cells vs propidium iodide staining intensity (DNA content per cell). In contrast to SEQ ID NO:4, but consistent with the lack of effect of SEQ ID NO:2 treatment on HeLa cell proliferation (FIG. 2A), the cell cycle distribution of HeLa cells was similar in cells treated with control SEQ ID NO:3 (FIG. 10A) and SEQ ID NO:2 (FIG. 10B).

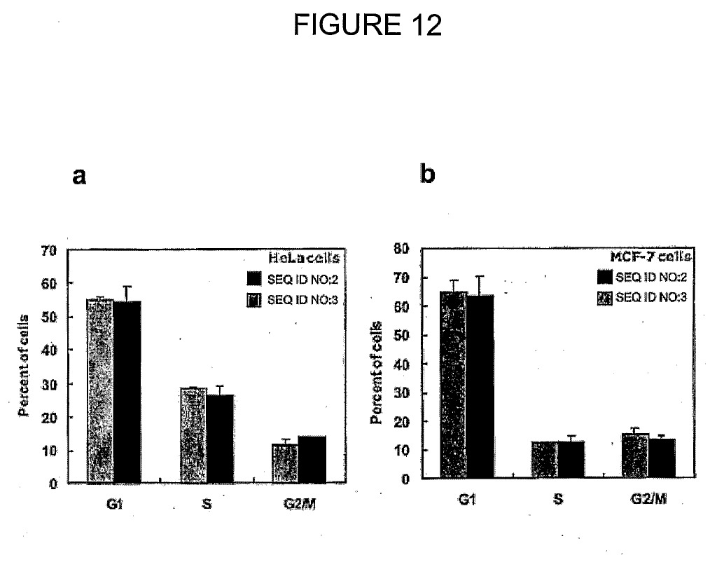

FIG. 12A shows the percent of cells in each of G1, S and G2/M stages of the cell cycle for oligonucleotide-treated HeLa (Panel A) and MCF-7 (Panel B) cells. No difference was observed between cells treated with SEQ ID NO:2 and control SEQ ID NO:3 in either cell line.

The data provided in Examples 1-6 indicates that antisense-mediated reduction in TS mRNA level (resulting in reduced TS protein levels) does not necessarily predict the capacity of antisense oligonucleotides targeting TS to inhibit cell proliferation. The differential effects demonstrated by SEQ ID NO:2 in different cell types suggest that this oligonucleotide may affect physiological processes other than TS protein production that impact proliferation and apoptosis. While it is formally possible that these SEQ ID NO:2 interacts with target RNAs other than TS mRNA, or with unidentified proteins through aptameric interactions; database queries predict no interactions with other known mRNA sequences.

Real-time quantitative PCR showed similar basal levels of TS mRNA in untreated HeLa and MCF-7 cells, whereas western blot analysis indicated approximately a 40-fold higher abundance of TS protein in untreated HeLa compared with MCF-7 cells. Thus the multiple steps of post-transcriptional regulation of TS protein expression are activated to a different extent in each of these two cell lines. Apoptosis by SEQ ID NO:2 is likely mediated in MCF-7 cells through an interaction with TS mRNA or protein, and reducing TS mRNA and protein levels in MCF-7 cells may prevent apoptosis induced by subsequent SEQ ID NO:2 treatment. Regardless of the underlying mechanism of action, SEQ ID NO:2-induced apoptosis could be a therapeutically useful outcome, and we are also testing whether this novel pathway can be similarly activated in additional breast cancer cell lines.

The methods outlined above can be used to investigate whether SEQ ID NO:2, and other antisense oligonucleotides targeting the coding region of TS mRNA, shows similar effects in other breast cancer cell lines, as well as other cancer cell lines, such as colorectal and/or colon cancer cell lines.

The disclosure of all patents, publications, including published patent applications, and database entries referenced in this specification are specifically incorporated by reference in their entirety to the same extent as if each such individual patent, publication, and database entry were specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. An antisense oligonucleotide targeted to thymidylate synthase for use to inhibit the proliferation of cancer cells in a subject, said antisense oligonucleotide having a sequence between about 7 and about 50 nucleotides in length comprising 7 or more consecutive nucleotides complementary to the coding region of a human thymidylate synthase mRNA, wherein said antisense oligonucleotide inhibits the proliferation of said cancer cells without decreasing the level of thymidylate synthase mRNA in said cells.

2. The antisense oligonucleotide according to claim 1, wherein said coding region of a human thymidylate synthase mRNA has a sequence as set forth in SEQ ID NO: 1 from nucleotides 109 to 500.

3. The antisense oligonucleotide according to claim 1, wherein said coding region of a human thymidylate synthase mRNA has a sequence as set forth in SEQ ID NO: 1 from nucleotides 150 to 300.

4. The antisense oligonucleotide according to claim 1, wherein said antisense oligonucleotide comprises 7 or more consecutive nucleotides from SEQ NO:2.

5. The antisense oligonucleotide according to claim 1, wherein said antisense oligonucleotide has a sequence as set forth in any one of SEQ NOs:2, 7, 8 or 9.

6. The antisense oligonucleotide according to claim 1, wherein said antisense oligonucleotide is provided as the antisense strand of a siRNA molecule.

7. The antisense oligonucleotide according to claim 1, wherein said cancer cells are solid tumour cells.

8. The antisense oligonucleotide according to claim 1, wherein said cancer cells are breast cancer cells.

9. An antisense oligonucleotide targeted to thymidylate synthase for use to induce apoptosis in cancer cells in a subject, said antisense oligonucleotide having a sequence between about 7 and about 50 nucleotides in length comprising a sequence of 7 or more consecutive nucleotides complementary to the coding region of a human thymidylate synthase mRNA, wherein said antisense oligonucleotide induces apoptosis of said cancer cells without decreasing the level of thymidylate synthase mRNA in said cells.

10. The antisense oligonucleotide according to claim 9, wherein said coding region of a human thymidylate synthase mRNA has a sequence as set forth in SEQ ID NO: 1 from nucleotides 109 to 500.

11. The antisense oligonucleotide according to claim 9, wherein said coding region of a human thymidylate synthase mRNA has a sequence as set forth in SEQ ID NO:1 from nucleotides 150 to 300.

12. The antisense oligonucleotide according to claim 9, wherein said antisense oligonucleotide comprises 7 or more consecutive nucleotides from SEQ ID NO:2.

13. The antisense oligonucleotide according to claim 9, wherein said antisense oligonucleotide has a sequence as set forth in any one of SEQ ID NOs:2, 7, 8 or 9.

14. The antisense oligonucleotide according to claim 9, wherein said antisense oligonucleotide is provided as the antisense strand of a siRNA molecule.

15. The antisense oligonucleotide according to claim 9, wherein said cancer cells are sold tumour cells.

16. The antisense oligonucleotide according to claim 9, wherein said cancer cells are breast cancer cells.

17. An antisense oligonucleotide having a sequence between about 7 and about 50 nucleotides in length comprising a sequence of 7 or more consecutive nucleotides complementary to the coding region of a human thymidylate synthase mRNA, wherein said antisense oligonucleotide inhibits proliferation of cancer cells without decreasing the level of human thymidylate synthase mRNA in said cells.

18. The antisense oligonucleotide according to claim 17, wherein said antisense oligonucleotide induces apoptosis in said cancer cells.

19. The antisense oligonucleotide according to claim 17, wherein said coding region of a human thymidylate synthase mRNA has a sequence as set forth in SEQ ID NO:1 from nucleotides 109 to 500.

20. The antisense oligonucleotide according to claim 17, wherein said coding region of a human thymidylate synthase mRNA has a sequence as set forth in SEQ ID NO:1 from nucleotides 150 to 300.

21. The antisense oligonucleotide according to claim 17, wherein said antisense oligonucleotide comprises 7 or more consecutive nucleotides from SEQ ID NO:2.

22. The antisense oligonucleotide according to claim 17, wherein said antisense oligonucleotide has a sequence as set forth in any one of SEQ ID NOs:2, 7, 8 or 9.

23. The antisense oligonucleotide according to claim 17, wherein said antisense oligonucleotide forms the antisense strand of a siRNA molecule.

24. The antisense oligonucleotide according to claim 17, wherein said cancer cells are breast cancer cells.

25. A method of inhibiting the proliferation of cancer cells in a subject, said method comprising contacting said cells with an effective amount of an antisense oligonucleotide targeted to thymidylate synthase, said antisense oligonucleotide having a sequence between about 7 and about 50 nucleotides in length comprising a sequence of 7 or more consecutive nucleotides complementary to the coding region of a human thymidylate synthase mRNA, wherein said antisense oligonucleotide inhibits the proliferation of said cancer cells without decreasing the level of thymidylate synthase mRNA in said cells.

26. The method according to claim 25, wherein said coding region of a human thymidylate synthase mRNA has a sequence as set forth in SEQ ID NO:1 from nucleotides 109 to 500.

27. The method according to claim 25, wherein said coding region of a human thymidylate synthase mRNA has a sequence as set forth in SEQ NO:1 from nucleotides 150 to 300.

28. The method according to claim 25, wherein said antisense oligonucleotide comprises 7 or more consecutive nucleotides from SEQ ID NO:2.

29. The method according to claim 25, wherein said antisense oligonucleotide has a sequence as set forth in any one of SEQ ID NOs:2, 7, 8 or 9.

30. The method according to claim 25, wherein said antisense oligonucleotide is provided as the antisense strand of a siRNA molecule.

31. The method according to claim 25, wherein said cancer cells are solid tumour cells.

32. The method according to claim 32, wherein said cancer cells are breast cancer cells.

33. A method of increasing apoptosis in cancer cells in a subject comprising contacting said cells with an effective amount of an antisense oligonucleotide targeted to thymidylate synthase, said antisense oligonucleotide having a sequence between about 7 and about 50 nucleotides in length comprising a sequence of 7 or more consecutive nucleotides complementary to the coding region of a human thymidylate synthase mRNA, wherein said antisense oligonucleotide inhibits the proliferation of said cancer cells without decreasing the level of thymidylate synthase mRNA in said cells.

34. The method according to claim 33, wherein said coding region of a human thymidylate synthase mRNA has a sequence as set forth in SEQ ID NO:1 from nucleotides 109 to 500.

35. The method according to claim 33, wherein said coding region of a human thymidylate synthase mRNA has a sequence as set forth in SEQ ED NO:1 from nucleotides 150 to 300.

36. The method according to claim 33, wherein said antisense oligonucleotide comprises 7 or more consecutive nucleotides from SEQ ID NO:2.

37. The method according to claim 33, wherein said antisense oligonucleotide has a sequence as set forth in any one of SEQ ID NOs:2, 7, 8 or 9.

38. The method according to claim 33, wherein said antisense oligonucleotide is provided as the antisense strand of a siRNA molecule.

39. The method according to claim 33, wherein said cancer cells are solid tumour cells.

40. The method according to claim 33, wherein said cancer cells are breast cancer cells.

41. A method of treating cancer in a subject in need thereof comprising administering to said subject an effective amount of the antisense oligonucleotide according to claim 17.


SEQUENCE LISTING (9 sequences)

SEQ ID NO: 1 - DNA, 1536 bp, Homo sapiens
  misc_feature (1)..(1536): Human thymidylate synthase mRNA
ggggggggggggaccacttggcctgcctccgtcccgccgcgccacttggcctgcctccgtcccgccgcgccacttcgcctgcctccgtcccccgcccgccgcgccatgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgaccgcacgggcaccggcaccctgtcggtattcggcatgcaggcgcgctacagcctgagagatgaattccctctgctgacaaccaaacgtgtgttctggaagggtgttttggaggagttgctgtggtttatcaagggatccacaaatgctaaagagctgtcttccaagggagtgaaaatctgggatgccaatggatcccgagactttttggacagcctgggattctccaccagagaagaaggggacttgggcccagtttatggcttccagtggaggcattttggggcagaatacagagatatggaatcagattattcaggacagggagttgaccaactgcaaagagtgattgacaccatcaaaaccaaccctgacgacagaagaatcatcatgtgcgcttggaatccaagagatcttcctctgatggcgctgcctccatgccatgccctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaagccaggtgactttatacacactttgggagatgcacatatttacctgaatcacatcgagccactgaaaattcagcttcagcgagaacccagacctttcccaaagctcaggattcttcgaaaagttgagaaaattgatgacttcaaagctgaagactttcagattgaagggtacaatccgcatccaactattaaaatggaaatggctgtttagggtgctttcaaaggagcttgaaggatattgtcagtctttaggggttgggctggatgccgaggtaaaagttctttttgctctaaaagaaaaaggaactaggtcaaaaatctgtccgtgacctatcagttattaatttttaaggatgttgccactggcaaatgtaactgtgccagttctttccataataaaaggctttgagttaactcactgagggtatctgacaatgctgaggttatgaacaaagtgaggagaatgaaatgtatgtgctcttagcaaaaacatgtatgtgcatttcaatcccacgtacttataaagaaggttggtgaatttcacaagctatttttggaatatttttagaatattttaagaatttcacaagctattccctcaaatctgagggagctgagtaacaccatcgatcatgatgtagagtgtggttatgaactttatagttgttttatatgttgctataataaagaagtgttctgc

SEQ ID NO: 2 - DNA, 20 bp, Artificial Sequence
  Oligonucleotide complementary to bases 214 to 233 within the human thymidylate synthase mRNA coding region
cagcggaggatgtgttggat

SEQ ID NO: 3 - DNA, 20 bp, Artificial Sequence
  control scrambled oligonucleotide
cggcacgcccataggcggcg

SEQ ID NO: 4 - DNA, 20 bp, Artificial Sequence
  Oligonucleotide complementary to bases 1184 to 1203 within the human thymidylate synthase mRNA 3′-UTR
gccagtggcaacatccttaa

SEQ ID NO: 5 - DNA, 21 bp, Artificial Sequence
  control scrambled oligonucleotide
atggcgccaacggttcctaaa

SEQ ID NO: 6 - DNA, 20 bp, Artificial Sequence
  control scrambled oligonucleotide
ggagtgcgtgagtcgatgta

SEQ ID NO: 7 - DNA, 19 bp, Artificial Sequence
  Oligonucleotide complementary to bases 121 to 140 within the human thymidylate synthase mRNA coding region
gccggcgcggcagctccga

SEQ ID NO: 8 - DNA, 20 bp, Artificial Sequence
  Oligonucleotide complementary to bases 111 to 130 within the human thymidylate synthase mRNA coding region
catgccgaataccgacaggg

SEQ ID NO: 9 - DNA, 20 bp, Artificial Sequence
  Oligonucleotide complementary to bases 269 to 278 within the thymidylate synthase mRNA coding region
catgccgaataccgacaggg